(19) United States
(12) Patent Application Publication
(10) Pub. No.: US 2026/0308214 A1
(43) Pub. Date: Oct. 8, 2026

(54) THERAPEUTIC COMPOSITION

(71) Applicant: KING'S COLLEGE LONDON, London (GB)

(72) Inventors: Sara GAMEA, London (GB); Sherif ELSHARKAWY, London (GB)

(21) Appl. No.: 19/483,907

(22) PCT Filed: May 10, 2024
(86) PCT No.: PCT/EP2024/062990

§ 371 Date: Nov. 12, 2025

(30) Foreign Application Priority Data
May 12, 2023 (GB) 2307118.6

(51) Int. Cl. C07K 14/47; A61K 38/00; A61L 27/22

(52) U.S. Cl. CPC C07K 14/4741; A61L 27/227; A61K 38/00; A61L 2430/12

(57) ABSTRACT

This invention relates to compositions comprising keratins, to methods of producing such compositions and to their use in various therapeutic applications, particularly in dentistry and bone repair. In particular, it relates to keratin compositions produced by dissolving keratins in a solvent, casting the resulting solution and allowing it to dry.

### TECHNICAL FIELD

This invention relates to compositions comprising keratins, to methods of producing such compositions and to their use in various therapeutic applications, particularly in dentistry and bone repair.

### BACKGROUND

Dental caries is the most prevalent chronic disease worldwide that affects dental hard tissues. Caries accounts for almost half of all tooth extractions and therefore remains a major public health problem. It affects both primary and permanent teeth. It ranges from mild white spot lesions (WSL) to severe loss of tooth structure, requiring extensive restorative treatment.¹ Amelogenin, an intrinsically disordered protein (IDP), is the major extracellular protein in developing enamel and is thought to be critical for the formation of the hierarchical architecture of enamel.² The inventors have used keratins as models of IDPs to develop clinically-friendly approaches to treat WSL. In particular, the inventors optimised the keratin extraction from natural waste tissues including sheep wool and human hair, developed crosslinking approaches specific for keratins to optimise their protein disorder-order synergism, and infiltrated WSL in situ, and subsequently mineralised the keratins scaffolds.

Biomineralization is the process by which living organisms produce minerals under the control of an organic matrix that nucleates and directs the hierarchical growth and morphogenesis of mineralized tissue. The study of biomineralization is crucial not only for gaining a better knowledge of how mineral-rich tissues are created in vivo, but also for providing inspiration for advanced material design.³ Biomimetic reconstruction of dental enamel may be an ideal method for regrowing ordered enamel-resembling the apatite crystals with robust adhesion to the natural enamel surface as an alternative to traditional treatments. Such an approach will lead to a strong tooth surface and will eliminate the problem of secondary caries. Biomimetic strategies for enamel repair have therefore attracted increasing interest in materials science and dentistry and are widely considered to be promising approaches to the prevention, restoration, and treatment of defective enamel.

During the biomineralization of hard tissue, the microstructures of natural materials are precisely controlled and duplicated.² Enamel formation, or amelogenesis, is a highly regulated process involving precise genetic control as well as protein-protein interactions, protein-mineral interactions, and interactions involving the cell membrane. The microstructures of natural materials are accurately regulated and replicated during the biomineralization of hard tissue. Amelogenesis, or the creation of enamel, is a highly regulated process including precise genetic control as well as protein-protein, protein-mineral, and cell membrane interactions.² The inventors have identified that proteins matrices can act as templates to derive the nucleation of hydroxyapatites in the mineralized tissues and mimic the biomineralization frontier to induce the hierarchical regeneration of enamel.

Despite several attempts at enamel remineralization and restoration of enamel structure and characteristics using diverse techniques, no clinically useful repair has been found. This is, at least in part, owing to the fact that natural enamel's complex hierarchical structure cannot be duplicated on a wide scale in laboratories. Non-invasive remineralisation strategies of early white-spots lesions on tooth surfaces using biomimetic approaches were attempted by Kirkham et al.⁴, 2007 who studied a self-assembling peptide scaffold known as Curodont™, which induces the nucleation, but was shown to exhibit poor crystalline hydroxyapatite. Chen et al.⁵, 2006 also developed a wet chemical method to synthesise the prism-like and crystal structures of enamel but with dimensions that are far bigger than those of natural enamel and only at high temperatures and pressures which are inapplicable clinically. Yamagishi et al.⁶ and Yin et al.⁷, have developed inorganic chemical methods to grow aligned enamel-like apatite nanocrystals on dental enamel but were unable to create the crucial hierarchical architecture. Shao et al.⁸ were also successful in developing inorganic chemical methods to grow aligned enamel-like apatite nanocrystals, which can establish a biomimetic crystalline-amorphous mineralization frontier to induce the epitaxial growth of enamel; however, only limited enamel thickness was regenerated.

Also, work by Oldak et al.⁹ using amelogenin-containing chitosan hydrogel for enamel reconstruction through amelogenin supramolecular assembly, stabilizing Ca—P clusters was found to produce a dense interface between the newly grown layer. Natural enamel was formed and bundles of organized crystals were found inside the repaired layer. However, the development of organized apatite nanocrystals with the distinctive hierarchical order was not attained. Recently, Elsharkawy et al.¹⁰ developed a material that mimics the structural hierarchical features of dental enamel. They reported a protein mediated mineralization process that takes advantage of disorder-order interplay using elastin-like recombinamers. Although this technology was found to produce mineralized structures that greatly resemble the enamel hierarchical structure and represents a potential strategy for designing materials that may open opportunities for hard tissue repair, it is not clinically applicable.

Protein-based biomaterials have shown significant potential in a variety of chemical and biological applications, due to their capacity to serve as synthetic extracellular matrixes that promote cell-cell and cell-matrix interactions. Proteinaceous macromolecules are extremely biocompatible and have a variety of physiochemical characteristics and bioactivities, making them especially appealing for medicinal applications. IDPs have been known to play a fundamental role in mineralization. IDPs contribute to intermolecular interactions at the protein-mineral interface.¹¹ Beniash et al.¹² reported that when amelogenin, a highly conserved IDP, interacts with the developing endocrine system, it undergoes a conformational transition from disordered random coils to organised-sheet structures. During enamel development, this conformational shift is known to guide crystal growth¹³. Carneiro et al.¹⁴ also found that the unique hierarchical structure of mature enamel may necessitate amelogenin conformational arrangement into amyloid-like nanoribbons.

To sum up, many attempts have explored the possibilities of synthesising enamel-like tissues, however reconstructing enamel at all the length-scales is a challenging task. The invention recreates highly ordered mineralised structures by using keratin-based hydrogels. Keratin represents the most abundant structural proteins in epithelial cells and is considered one of the most important biopolymers in animals.¹⁵ Keratinous materials have a high content of cysteine that distinguishes them from other proteins, and are typically durable, tough and unreactive to the natural environment; they are assumed to provide mechanical support. They have a strong potential in the field of biomaterial research due to their intrinsic ability to self-assembly, biocompatibility, biodegradation, mechanical longevity and support cellular proliferation. These biomedical capabilities are due to the highly ordered hierarchical structure of the keratin fibres, starting from the nano-scaled intermediate filaments to the micro-scaled cortical cells, and their aptness of self-assembly allows them to polymerize into porous scaffolds.^{16,17}

The inventors have produced composition useful in treating dental caries amongst other conditions and have developed new production methods.

### SUMMARY OF THE INVENTION

According to the invention, there is provided a method of producing a keratin composition, particularly a keratin membrane, comprising the steps of:
(a) Providing a keratin solution comprising keratin in a solvent;
(b) Casting the solution on a substrate; and
(c) Allowing the solution to dry.

The solution comprises one, or preferably at least two keratins. Keratins are proteins found in animal and human hair, fur and other tissues. Different keratins are well known in the art. For example, known human keratins include KRT23, KRT24, KRT25, KRT26, KRT27, KRT28, KRT31, KRT32, KRT33A, KRT33B, KRT34, KRT35, KRT36, KRT37, KRT38, KRT39, KRT40, KRT71, KRT72, KRT73, KRT74, KRT75, KRT76, KRT77, KRT78, KRT79, KRT8, KRT80, KRT81, KRT82, KRT83, KRT84, KRT85 and KRT86.

In a particular embodiment, the keratin or keratins are wool keratins. Wool keratins include keratin intermediate filaments (KIFs), S-carboxy methyl kerateine A (SCMK-A) and S-carboxy methyl kerateine B (SCMK-B), the keratin-associated proteins (KAPs) e.g.: KAP1-1, KAP2, KAP3, KAP4, KAP5, KAP6, KAP7, KAP8, KAP11, KAP13, KAP24, inner root sheet type I keratins: oIRSa1, oIRSa2, oIRSa3-1, and oIRSa3.2

When the solution comprises two or more keratins, the keratins are preferably of differing molecular weight, or are encoded by different keratin genes, or differ by way of modifications made to them. When the solution comprises one keratin, the keratin is preferably not KR75 (or KRT75). KR75 (also known as K6 hf) is known to form within the inner sheath of hair follicles, the nail bed and lingual papillae in humans. KR75 is an isoform of keratin 6 and has a molecular weight of 59.5 kDa and an isoelectric pH of 7.9. In nature the keratin filaments containing KR75 are oriented perpendicularly to the longitudinal axis of the hair like the ‘hoops of a barrel’.

When the solution comprises more than one keratin, a high level of variation in the keratins within a solution or structure is preferable. For example, in one preferred embodiment the lightest keratin present in the solution or structure has a molecular weight that is less than 5%, less than 10%, less than 15%, less than 20% less than 25%, less than 30%, less than 35%, less than 40%, less than 45%, less than 50%, less than 55% or less than 60% of the molecular weight of the heaviest keratin present in the solution or structure.

The solution may comprise at least three, at least four, at least five, at least six, at least seven, at least eight, at least nine or at least ten keratins.

Optionally, one, both, several or all of the keratins may be highly negative. The term highly negative keratin would be understood by one skilled in the art to mean a keratin comprising a plurality of negatively charged functional groups, such as COOH. Preferably at least one of the keratins present in the keratin solution or keratin structure is highly negative. By “highly negative” it is meant that the keratin has a pKₐ of less than 7.

As is known in the art, zeta (ζ) potential can be used as an indicator of pKₐ with respect to complex molecules, such as keratin. In one embodiment, at least one of the keratins present in the keratin solution or keratin structure has a zeta potential of at least −2 mV at physiological pH. In one embodiment, at least one of the keratins present in the keratin solution or keratin structure has a zeta potential of at most −40 mV at physiological pH. Preferably at least one of the keratins present in the keratin solution or keratin structure has a zeta potential of at least −4 mV at physiological pH. More preferably at least one of the keratins present in the keratin solution or keratin structure has a zeta potential of at least −6 mV at physiological pH. More preferably at least one of the keratins present in the keratin solution or keratin structure has a zeta potential of at least −8 mV at physiological pH. More preferably at least one of the keratins present in the keratin solution or keratin structure has a zeta potential of at least −10 mV at physiological pH.

Optionally at least one, or both, several or all of the keratins are alpha-keratins. Optionally one or more of the keratins are beta-keratins. Alpha keratins are found in all vertebrates, and form the hair (including wool), the outer layer of skin, horns, nails, claws, hooves and baleen plates of mammals. Beta-keratins are found only in living reptiles and birds, in particular in nails, scales, claws, shells, feathers and beaks. Optionally, the keratin solution and structure of the present invention may be derived from any such keratin source.

Optionally, at least one, both, several or all of the keratins are high sulphur keratins. Optionally, at least one, both, several or all of the keratins are low sulphur keratins. Preferably, the solution comprises both high and low sulphur keratins. The terms high sulphur keratin and low sulphur keratins are well known in the art.

High sulphur keratins can generally be separated into two groups, one of which contains proteins with cysteine levels (measured as S-carboxymethyl cysteine or SCMC) of more than 30mole %, known as ultrahigh sulphur (UHS), and the other of which contains proteins with high glycine and/or tyrosine levels (HGT).

Keratins, particularly low sulphur types, are known to contain significant amounts of aspartic (ASP) and glutamic (Glu) acid. Without being bound by theory, the abundance of negatively charged functional groups, such as COOH in aspartic acid (Asp) and glutamic acid (Glu), is thought to enhance template-directed calcium and phosphate precipitation due to their ability to attract calcium (Ca²⁺) and (PO4³⁻) ions. It is thought that hydroxyapatite nucleation is initiated during the bone mineralization regulation process by a group of negatively charged phosphorylated non-collagenous proteins that attract Ca²⁺ and PO4³⁻ ions through their charged amino acid (AA) domains and increase local supersaturation to a level sufficient to form nuclei of a critical size, which can develop into hydroxyapatite (HA) crystals. In addition to their role in mineralization, charged AAs such as Asp, and Glu bound to HA surface were also shown to promote protein absorption and osteoblast proliferation. (Song J, Malathong V, Bertozzi C R. 2005 Mineralization of synthetic polymer scaffolds: a bottom-up approach for the development of artificial bone. J. Am. Chem. Soc. 127, 3366-3372, George A, Veis A. 2008 Phosphorylated proteins and control over apatite nucleation, crystal growth, and inhibition. Chem. Rev. 108, 4670-4693, Tavafoghi, M., & Cerruti, M. (2016). The role of amino acids in hydroxyapatite mineralization. Journal of The Royal Society Interface, 13(123), 20160462).

Various solvents are useful in the invention. For example, the solvent used may be water, one or more polar aprotic solvent (for example dimethyl sulfoxide or dimethylformamide), or one or more alcohol (for example ethanol), or a combination thereof. Optionally, the solvent may be ethanol, water, DMSO or DMF, or a combination of two or more thereof.

For example, the solvent may comprise or be:
water;
Ethanol;
Water and ethanol;
Water and DMF;
DMSO;
DMSO and DMF.

Where two or more solvents are used, they may be used in equal proportions or in different proportions. For example, the solvent may be 50:50 DMSO and DMF. Optionally, the solvent may comprise 10, 20, 30, 40, 50, 60, 70, 80 or 90% water. Optionally, the solvent may comprise 10, 20, 30, 40, 50, 60, 70, 80 or 90% DMSO. Optionally, the solvent may comprise 10, 20, 30, 40, 50, 60, 70, 80 or 90% DMF.

The solvent may be deionised and/or comprise essentially no solutes other than keratin. Alternatively salts such as sodium, sulphate, chloride, zinc, carbonate, phosphate, calcium, fluoride, iron or potassium may be added to the solvent. Such salts may aid the mineralisation process, which in turn can help with the bone or enamel regeneration process.

The pH of the solvent may be between 3 and 12, or between 3.5 and 12, or between 4 and 12, or between 4.5 and 12, or between 5 and 12, or between 5.5 and 12, or between 6 and 12, or between 6.5 and 12, or between 7 and 12, or between 6.5 and 11.5 or between 7 and 11.5 or between 7 and 11 or between 4 and 11, or 5 and 11. The pH of the solvent is preferably at least 4, or at least 4.5, or at least 5, or at least 5.5, 6, or at least 6.25, or at least 6.5, or at least 6.75 or at least 7. The pH of the solvent is preferably less than 12, or less than 11.75, or less than 11.5, or less than 11.25 or less than 11.

The concentration of the keratin in the solution is preferably between 1 and 25 wt %, or between 1 and 20 wt %, or between 1 and 15 wt %, or between 1 and 12.5 wt % or between 1 and 10 wt %, or between 2 and 25 wt %, or between 2 and 20 wt %, or between 2 and 15 wt % or between 2 and 12.5 wt % or between 2 and 10 wt %, or between 3 and 25 wt %, or between 3 and 20 wt %, or between 3 and 15 wt % or between 3 and 12.5 wt % or between 3 and 10 wt %, or between 4 and 25 wt %, or between 4 and 20 wt %, or between 4 and 15 wt % or between 4 and 12.5 wt % or between 4 and 10 wt % or between 5 and 25 wt %, or between 5 and 20 wt %, or between 5 and 15 wt % or between 5 and 12.5 wt % or between 5 and 10 wt %.

The keratin concentration is preferably at least 0.5 wt %, or at least 1 wt %, or at least 1.5 wt %, or at least 2 wt %, or at least 2.5 wt %, or at least 3 wt %, or at least 3.5 wt %, or at least 4 wt % or at least 4.5 wt %, or at least 5 wt %.

The keratin concentration is preferably less than 25 wt %, or less than 20 wt %, or less than 15 wt %, or less than 14 wt %, or less than 13 wt %, or less than 12 wt %, or less than 11 wt %, or less than 10 wt %.

In one embodiment, the keratin solution has a neutral pH. In one embodiment, the pH of the keratin solution is between 5.5 and 8.5, between 6.0 and 8.0, between 6.5 and 7.5, between 6.6 and 7.4, between 6.7 and 7.3, between 6.8 and 7.2, between 6.9 and 7.1 or preferably around 7 or at 7.″

Casting means applying the keratin solution onto a substrate. The solution may then be allowed to dry to form a structure, such as a membrane. Any appropriate substrate may be used in step (b). In some embodiments, the substrate is at least slightly hydrophobic, to allow the resulting membrane to be easily removed. Optionally, the substrate may be a polymeric organosilicon compound, such as polymethylsiloxane or polydimethylsiloxane. In an embodiment, the substrate is a tooth or teeth.

Casting may be carried out in vitro or ex vivo or in vitro.

The method may comprise repeating steps (b) and optionally (c) to form different shaped or sizes structures.

For example, to form a membrane structure in vitro, the step of casting may comprise drop casting the solution onto a non-reactive hydrophobic surface as described. Thicker structures may be made through allowing the layer to dry and then casting a further layer over the top. This process can be repeated in order to obtain the desired thickness. 3D printing using the keratin solution can be used to automate this process.

After casting, the solution is allowed to dry. Optionally, the keratin solution may be dried for at least 30, 40, 50, 60, 75, 90, 105, 120 or 150 seconds. Optionally, it may be dried at a temperature between 15° C. and 50° C., or between 15° C. and 45° C. or between 15° C. and 40° C. or between 15° C. and 38° C. Optionally, the solution may be dried at a temperature of at least 10, 12, 15, 17, 18, 19, 20, 22, 25, 27, 30, 32, 35, 37, 40, 42 or 45° C. Optionally, The solution may be dried at a temperature of less than 55, 52, 50, 47, 45, 42, 40, 37, 35, 32, 30, 27 or 25° C.

Drying may be carried out in vitro or ex vivo or in vitro.

The solution may also comprise a cross-linker, such as triethylene glycol-dimethacrylate (TEGDMA), hexamethylene diisocyantate (HDI) and/or poly-ethylene glycol dimethacrylate (PEGDMA). Optionally, the cross-linker may include a photo-initiator, such as lithium phenyl-2,4,6-trimethylbenzoylphosphinate (LAP), which may be used to speed up drying time. Other photo-cross-linkers that may be used include trimethylbenzoyl-diphenylphosphine oxide (TPO), benzoyl peroxide (BPO), 2,2-Dimethoxy-2phenylacetophenone (DMPA), camphorquinone (CQ), phenanthrenequinone (PQ), benzophenone (BP), and 1-phenyl-1,2 propanodione (PPD).

The cross-linker may be used at a particularly molar ratio to the average lysine and/or cysteine residues in the keratins. For example, the TEGDMA may be used at a ratio of around 1, 10, 20, 30, 40 or 50 to one lysine and/or cysteine residue. The HDI may be used at a ratio of around 1, 2, 3, 5, 8 or 10 to one lysine and/or cysteine residue.

The method may comprise the step of directing light at the solution, after casting it, in order to photo-initiate membrane formation.

The solution generally dries to form a membrane. The membrane comprises spherulites, which are generally Maltese Cross in shape. The spherulites are generally thought of as crystalline aggregates of closely packed chains that can provide the protein with significant strength and rigidity. The spherulites are generally between 3 and 50 μm in diameter. Preferably, the spherulites are between 1 and 10 μm in diameter. Preferably, the spherulites are at least 3, 4, 5, 6, 7, 8 μm in diameter. The spherulites are preferably less than 50, 45, 40, 35 and 30 μm in diameter. Where the composition is in the form of a membrane, the membrane optionally comprises at least 10, 15, 20, 25 or 30 spherulites/mm². Where the composition is in the form of a membrane, the membrane optionally comprises less than 120, 115, 110, 105 or 100 spherulites/mm². Where the composition is in the form of a membrane, the membrane is optionally at least 10, 15, 20, 25 or 30 μm. Where the composition is in the form of a membrane, the membrane is optionally less than 230, 225, 220, 215, 210, 205 or 200 μm thick.

Spherulites are known to those skilled in the art. Crystal lamellae self-assemble into spherical superstructures where highly ordered lamellar sheets grow out radially from a centre. Without wishing to be bound by theory, it is thought that these spherulites act as nucleation points that promote hierarchical mineral growth, which aids enamel or bone regeneration. Alignment of the keratin within the lamellae results in birefringence, which leads to the “Maltese cross” pattern when the structure is viewed between crossed polarisers with an optical microscope. As such, it is very straightforward for the skilled person to determine whether or not a keratin structure comprises spherulite structures.

In one embodiment, the membrane comprises one or more large globules made of two or more, three or more, four or more, five or more, ten or more, twenty or more or fifty or more spherulites. In one, embodiment, the membrane comprises two or more, three or more, four or more, five or more, ten or more, twenty or more or fifty or more spherulites, wherein the spherulites aligned into strands.

In one embodiment, the keratin spherulites comprise β-sheets and random coil structures. In a preferred embodiment, the keratin spherulites comprise a higher proportion of β-sheets.

In one embodiment, the dried keratin membrane comprises an organic network of nanospheres on its surface. In a preferred embodiment, the nanospheres have diameters ranging between 10 and 60 nm, 20 and 50 nm or preferably 20 and 40 nm.

In one embodiment the dried keratin membrane comprises nano- and/or micro-fibrillar structures. In a particularly preferred embodiment, the fibrils are well-organized and aligned substantially parallel to one another. In one embodiment, the dried keratin membrane comprises a first and a second population of fibrils. In one embodiment, the fibrils in the first population have a width of 50 to 150 nm, 60 to 140 nm, 70 to 130 nm, 80 to 120 nm, 90 to 110 nm, or preferably around 100 nm. In one embodiment, the fibrils in the second population have a width of 400 to 600 nm, 450 to 550 nm, 470 to 530 nm, 480 to 520 nm, 490 to 510 nm, or preferably around 500 nm.

The step of providing the keratin solution may comprise the step of dissolving the keratin in the solvent. The keratin may be provided in any form suitable for dissolving the solvent. In particular, the keratin may be lyophilised. The keratin may be in the form of a dry powder.

The method may comprise the step of washing the composition, especially in water, or storing the composition, especially in water.

The method may comprise the step of
(d) Incubating the keratin composition in a mineralisation solution.

Incubating the composition generally results in the production of a mineralised keratin composition.

The mineralisation solution generally comprises calcium or a sodium salt or both. For example, the mineralisation solution may comprise calcium phosphate. It may also comprise sodium chloride or sodium fluoride. In an embodiment, the mineralisation solution comprises between about 0.1 mM and 1 M Ca²⁺, and/or between about 0.1 mM to about 1 M PO4³⁻. Optionally, it may contain from about 0.01 mM to about 1M F⁻. Optionally, it may contain zinc, barium, strontium, lead, silver, potassium, carbonate, iron, and/or magnesium.

An acid, such as nitric acid hydrochloric acid or phosphoric acid, may be added to the mineralisation solution to enable the calcium phosphate to dissolve. Accordingly, the mineralisation solution may comprise an acid. Optionally, the keratin composition may be incubated in the mineralisation solution at a pH of around 4, 5, 6 or 7.

Optionally, the keratin composition may be incubated in the mineralisation solution for at least 1, 2, 3, 4, 5, 6, 7, 8, 9, 10, 11, 12, 13 or 14 days. Optionally, the keratin composition may be incubated in the mineralisation solution for less than 14, 13, 12, 11, 10, 9, 8, 7, 6, 5, 4, 3 or 2 days Incubation may optionally take place at a temperature of between 15 and 90° C., or at least 15, 20, 25, 30, 35, 40, 45, 50, 55, 60° C., or less than 95, 90, 85, 80, 75, 70, 65, 60, 55 or 50° C., or at around 30, 35, 37, 40, 42 or 45° C. Preferably the incubation takes place at 37° C.

Following incubation, the method may comprise the step of removing the composition from mineralisation solution. It may be rinsed.

The mineralisation solution may be, or comprise a bodily fluid, such as saliva, blood, interstitial fluid, serum or plasma. Mineralisation may take place in vivo, ex vivo or in vitro. For example, mineralisation may take place in the mouth, with saliva as the mineralisation solution.

Alternatively, the mineralisation solution may be a hydroxyapatite solution. In a preferred embodiment the hydroxyapatite solution is a fluoride-rich hydroxyapatite solution, most preferably a supersaturated fluoride-rich hydroxyapatite solution.

The addition of the mineralisation solution to the membrane causes mineralisation to occur. The mineralised film comprises spherulite-like mineralized structures. In one embodiment, the mineralised film comprises two or more, three or more, four or more, five or more, ten or more, twenty or more or fifty or more spherulite-like mineralized structures. In one embodiment, the mineralized spherulitic structures have a spirally oriented morphology of assembled mineral nanocrystals. In one embodiment, the nanocrystals have a needle-like shape. In a preferred embodiment the nanocrystals comprise aligned prism-like structures.

In a preferred embodiment the provenance of the prism-like structures is the center of the spherulite, and the prism-like structures terminate on a point at the spherulite periphery.

Preferably the prism-like structures are 0.5 to 2 μm thick, 1.0 to 1.7 μm thick, or most preferably 1.360.33 μm thick. Preferably the prism-like structures are tens of micrometers in length. Preferably the prism-like structures have a diameter of 40 to 120 nm, 50 to 110 nm, 60 to 100 nm, or most preferably 83 #22.96 nm.

In one embodiment the prism-like structures are elongated apatite nanocrystals. In a preferred embodiment, the elongated apatite nanocrystals are aligned in a substantially parallel arrangement.

In a preferred embodiment, the spherulite-like mineralized structures comprise apatite crystals on both their surface and cross-section.

In one embodiment, the mineralised film has an increased hardness and/or increased Young's modulus, as compared with the film before the addition of the mineralisation solution. In one embodiment, the mineralised film has a Young's modulus of more than 5 GPa, more than 5.1 GPa, more than 5.5 GPa, more than 6 GPa, more than 6.5 GPa, more than 7 GPa, more than 7.5 GPa or more than 8.0 GPa. In one embodiment, the mineralised film has a hardness of more than 0.3 GPa, more than 0.4 GPa, more than 0.5 GPa, or more than 0.6 GPa.

In one embodiment the spherulite-like mineralized structures comprise a bottom layer, a top layer and an interface layer, wherein the interface layer lies between the bottom layer and the top layer.

In one embodiment, the bottom layer does not comprise apatite crystals. In a preferred embodiment, the bottom layer consists of, or substantially consists of, keratin organic matrix. In one embodiment, the bottom layer consists of, or substantially consists of, carbon and sulfur.

In one embodiment, the top layer comprises polycrystalline circular structures. In one embodiment, the top layer comprises calcium, phosphorous, sulfur, carbon and fluoride.

In one embodiment the interface layer comprises arched crystals. In one embodiment, the interface layer comprises fluoride. In one embodiment, the interface layer comprises the highest fluoride content of the three layers.

In one embodiment, the interface layer and/or top layer comprise nanospheres, wherein the nanospheres comprise, or substantially consist of, keratin organic matrix. In one embodiment, these nanospheres provide additional nucleation centres to promote the development of further nanocrystals.

In one embodiment, the mineralised spherulites comprise keratin rod-like (helical) structures.

There is also provided a keratin solution, comprising keratin and a solvent. The keratin solution and its components may be as described in relation to the method of the invention.

Optionally, the keratins for use in the method of the invention may be obtained by extracting keratin(s) from animal or human hair, fur, wool, hooves, shells, horns, claws, nails, quills or feathers. In particular, the keratins may be extracted from wool, such as wool from sheep, goats, camelids and rabbits, especially sheep.

There is provided a method of extracting keratin from animal or human tissue, such as hair, fur, wool, hooves, shells, horns, claws, nails, quills or feathers, particularly wool comprising the steps of:
(a) Mixing the tissue with an extraction solution;
(b) Heating the mixture;
(c) Separating a keratin containing supernatant from the remainder of the mixture.

The extraction solution optionally comprises one or more of urea, sodium dodecyl sulfate, 2-mercaptoethanol, thiourea dithiothreitol (DTT), [3-Cholamidopropyl)dimethylammonio]-1-propanesulfonate (CHAPS), L-cysteine, ampholytes, glycerol, triton X-100, sodium phosphate, sodium metabisulfite, sodium hydroxide, peracetic acid, 1-butyl-3-methylimidazolium chloride (BMIM+Cl₂), iodoacetate, zinc acetate, ethylenediaminetetraacetic acid (EDTA), trisaminomethane-hydrochloric acid (Tris-HCl) buffer, HEPES buffer and other buffers.

The mixture is optionally heated at between 4° and 60° C., or between 45 and 60° C., or between 4° and 55° C., or between 45 and 55° C. or at around 50° C., for around 8, 9, 10, 11, 12, 13 or 14 hours.

Optionally, the supernatant is separated from the mixture by filtration, centrifuging or a combination of the two.

The method may also comprise the step of dialysing the supernatant against water to produce a keratin solution.

The method may also comprise the step of drying the keratin solution, especially freeze drying it.

Also provided is a keratin composition, such as a membrane, comprising a keratin. In particular, the keratin composition comprises keratin spherulites. The composition may be mineralised. Aspects of the keratin composition may be as described in relation to the method of the invention. The keratin composition may be obtained by or obtainable by using the method of the invention.

Also provided is a pharmaceutical composition comprising a keratin composition or a mineralised keratin composition according to the invention. The pharmaceutical composition may further comprise a pharmaceutically acceptable carrier. Optionally, the pharmaceutical composition may be in the form of or added to a hydrogel, a solution, a paint, a varnish, a coating, a scaffold, such as a 3D printed scaffold, a membrane, a toothpaste, a strip, particularly a dental strip, a dental restorative material, a composite, a gum, a metallic implant, a cement, a ceramic, a paste, a malleable putty, a film or a dentine desensitizing agent.

The keratin solution or composition may also be used in the preparation of devices and structures for use in medical treatments. Also provided by the invention is a medical structure or device such as a synthetic graft, a prosthesis or orthosis, an implant or a tissue graft, particularly a dental or bone graft, comprising a keratin solution, a keratin composition, or a mineralised keratin composition or a pharmaceutical composition according to the invention.

Further provided is a keratin solution, a keratin composition, or a mineralised keratin composition, a pharmaceutical composition or a structure or device according to the invention, for use in therapy.

The solution, composition, structure or device may be for use in dental treatments, particularly for use in the treatment of WSLs or dental caries. In particular, it may be used to treat or prevent teeth demineralization, dental erosion, dental abrasion, alveolar bone erosion, periodontitis, peri-implantitis or dental pulp disease, dental hypersensitivity and soft tissue repair e.g., grafting and augmentation. Alternatively, it may be for use in bone repair, healing or regeneration.

In one embodiment, the solution, composition, structure or device is for use in the treatment or repair of defective enamel. In one embodiment, the solution, composition, structure or device stimulates enamel reconstruction. In a preferred embodiment, the reconstructed enamel has an aligned, oriented prism structure. In a preferred embodiment the reconstructed enamel has the same structure as the dental enamel of healthy teeth. In a preferred embodiment, the solution, composition, structure or device treats or repairs defective enamel across almost the whole depth of the lesion.

In one embodiment, the reconstructed enamel has a knoop hardness of more than 0.4 GPa, more than 0.5 GPa, more than 0.6 GPa, more than 0.7 GPa, more than 0.8 GPa, more than 0.9 GPa, more than 1.0 GPa, more than 1.1 GPa, more than 1.2 GPa, more than 1.3 GPa, more than 1.4 GPa, more than 1.5 GPa, more than 1.6 GPa, more than 1.7 GPa, more than 1.8 GPa, more than 1.9 GPa, or more than 2 GPa.

In one embodiment, the reconstructed enamel has a Young's modulus of more than 10 GPa, more than 15 GPa, more than 20 GPa, more than 25 GPa, more than 30 GPa, more than 35 GPa, more than 40 GPa, more than 45 GPa, more than 46 GPa, more than 47 GPa, more than 48 GPa, more than 49 GPa, more than 50 GPa, more than 51 GPa, more than 52 GPa, or more than 53 GPa.

In one embodiment, the reconstructed enamel has a hardness of more than 0.4 GPa, more than 0.5 GPa, more than 0.6 GPa, more than 0.7 GPa, more than 0.8 GPa, more than 0.9 GPa, more than 0.95 GPa, more than 1.0 GPa, or more than 1.05 GPa.

The solution, composition, structure or device may be for use in treating or preventing bone loss, in repairing damaged bone.

The solution, composition, structure or device may be for use in promoting or inducing bone healing, bone regeneration and/or osteogenesis. In one embodiment the solution, composition, structure or device may be for use in promoting or inducing bone healing, bone regeneration and/or osteogenesis of the skull. In one embodiment the solution, composition, structure or device may be for use in promoting or inducing bone healing, bone regeneration and/or osteogenesis of the calvarium.

The solution, composition, structure or device may be for use in the treatment of bone defects. In one embodiment the bone defect is the result of trauma, infection, congenital abnormalities, pathological diseases, and/or early tooth loss.

In a preferred embodiment solution, composition, structure or device allows for osteoconduction and/or osteoinductive signals that encourage osteogenesis and, in turn, induce biomineralization.

For example, the solution, composition, structure or device may be applied and cured during surgery, to replace a bone with an implant or to aid with mineralisation and subsequent healing.

Alternatively, the keratin solution may be dried into a specific shape that can then be applied to fill a cavity with a bone in order to aid remineralisation and healing of that bone. Scanning techniques known in art such as computerised tomography and magnetic resonance imaging, or surgical investigation, can be used to determine the shape of the bone cavity.

The solution, composition, structure or device may be for use in a single treatment, or multiple treatments, at the same site. In an embodiment, the solution, composition, structure or device is for regular use on teeth in order to promote or maintain a healthy enamel layer. In one embodiment, the solution, composition, structure or device is for regular application to healthy teeth in order to prevent enamel loss.

The solution or composition may be for use on its own, or in conjunction with one or more other active agents.

Further provided is a method of treating WSLs or dental caries, or bone damage or disorders, comprising administering a keratin composition, or a mineralised keratin composition or a pharmaceutical composition according to the invention, or utilising an implant or tissue graft of the invention.

Alternatively viewed, the present invention provides a use of the keratin solution or composition of the invention for the manufacture of a medicament for use in the treatment of any of the conditions described above.

In a yet further aspect the present invention provides a product or kit comprising (a) a keratin solution or composition according to the invention and (b) a further mineralisation solution and/or crosslinker. The product may be a combined preparation for separate, simultaneous, or sequential use in therapy.

The solution, composition, structure or device of the invention may be formulated and/or packaged for its intended use. For example, for dental applications, the solution or composition may be applied to the teeth with a brush like a paint or varnish. Alternatively, the solution or composition may be applied to the teeth via a dental tray. Such trays are commonly used in the art for, for example, applying solution for whitening the teeth. In a preferred embodiment, for dental applications, the solution may be applied as a spray. Optionally the spray is applied after the teeth have been cleaned, for instance by brushing. For bone defects, the solution or composition may be applied directly to a cavity in order for the structure to fill the cavity.

The invention further provides a kit comprising a keratin solution, keratin composition or pharmaceutical composition according to the invention and an applicator for applying the solution or composition to the site of interest. The applicator may, for example, be a brush, pad, spray, dropper or dental tray. The kit may further comprise instructions for use of the components.

It is envisaged that the solution, composition, structure or device of the present invention may be used with respect to any animal having teeth and/or bones. In one embodiment, the animal is a mammal, preferably a human.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is an illustration showing keratin extraction using a reduction process. Keratin source is harvested from, for example, wool (1). Keratin source is defatted by Soxhlet extraction (2). Keratin source is then filtered (3) and centrifuged (4). The supernatant is then isolated (5) and dialysed (6) leading to the keratin solution (7).

FIG. 2a is an illustration showing the fabrication steps of keratin membranes in vitro. After keratin extraction, the keratin is dissolved in one of Milli-Q water pH7-11, dimethyl sulfoxide (DMSO), or Dimethylformamide (DMF) (1). A crosslinker such as Tri-ethylene Glycol Dimethacrylate (TEGDMA), Hexamethylene diisocyanate (HDI) or poly-ethylene glycol dimethacrylate (PEGDMA) (with the addition of 3% wt lithium phenyl-2,4,6-trimethylbenzoylphosphinate (LAP) photo-initiator) may then be optionally added (2). The solution is then drop casted (3) onto a polydimethylsiloxane (PDMS) substrate (4) and left to dry.

FIG. 2b shows images of keratin membranes formed in vitro using various concentrations of keratin solution and using various concentrations of as tri-ethylene glycol dimethacrylate (TEGDMA) crosslinker. K₅ represents 5 wt % keratin solution, K₁₀ represents 10 wt % keratin solution. TE₁₀ represents a ratio of TEGDMA crosslinker to the lysine and cysteine amino-acid residues within the keratin of 1:10 (equivalent to a ratio of 100 μl keratin: 3.74 μl TEGDMA), TE₂₀ represents ratio of TEGDMA crosslinker to the lysine and cysteine amino-acid residues within the keratin of 1:20 (equivalent to a ratio of 100 μl keratin: 7.48 μl TEGDMA), and no mention of “TE” (i.e. “K₅” or “K₁₀”) indicates that no crosslinker is used.

Handling score: 1=difficult, 2=moderate, 3=easy. Collapse potential: 2=moderate, 3=low. Brittleness: 1=high, 2=moderate, 3=low. Opacity: 1=Opaque, 2=moderate opacity, 3=transparent.

FIG. 3 is SDS-PAGE of keratin aqueous solutions. FIG. 3a shows the bands at different concentrations for 2 different batches. Bands associated with low sulphur (LS) keratin (1), high sulphur (HSPs) keratin (2) and high glycine-tyrosine (HFT) keratin (3) are seen. FIG. 3b shows that artificially expressed keratin 75 (KR75) solutions result in a single band.

FIG. 4 is scanning electron microscopy images of keratin fibres observed in a keratin membrane fabricated with either 5 wt % keratin (a) or 10 wt % keratin (b) with triethylene glycol dimethacrylate (TEGDMA) crosslinker.

FIG. 5 are light microscopy images of keratin membranes. FIG. 5a is a polarized light microscopy image of a keratin membrane made by dissolving 5 wt % wool-extracted keratin with water only. Insert shows a higher magnification of an organic spherulite. FIG. 5b is a white light microscopy image of a keratin membrane made by dissolving 5 wt % keratin 75 (KR75) with water only.

FIG. 6 are scanning electron microscopy images of biomimetic mineralisation. FIGS. 6a to 6f show the membrane mineralisation stages on a membrane formed with 10 wt % keratin and triethylene glycol-dimethacrylate (TEGDMA) crosslinker (ratio of crosslinker to the lysine and cysteine amino-acid residues within the keratin of 1:10 (equivalent to a ratio of 100 μl keratin: 3.74 μl TEGDMA)) over a fourteen-day period. FIGS. 6g and 6h show cross-sections of the membrane, revealing the mineralised crystals. Images are also shown of keratin membranes fabricated with TEGDMA crosslinker (ratio of crosslinker to the lysine and cysteine amino-acid residues within the keratin of 1:10) at 5 wt % keratin (i) or 10 wt % keratin (j) with poly-ethylene glycol dimethacrylate (PEGDMA) crosslinker and the addition of 3% wt lithium phenyl-2,4,6-trimethylbenzoyl phosphinate (LAP) photo-initiator at 5% keratin (k) or with no crosslinker at 5% keratin (I).

FIG. 7 shows Hierarchical mineralization of keratin-based films. FIG. 7a is an image showing a Keratin film before mineralization. FIG. 7b is a polarized light microscope image of an organic keratin film depicting organic spherulites on its surface. The insert shows high magnification of a keratin spherulites with the characteristic birefringent Maltese-cross appearance. FIG. 7c scanning electron microscopy (SEM) image showing an organic spherulite before mineralization. FIG. 7d SEM demonstrating the self-assembly of the keratin into densely packed nanofibrils. FIG. 7e AFM image showing the nanofibrils in a 3D aspect. FIG. 7f SEM of a mineralized keratin film showing mineralized spherulites growing on the surface. The insert displays an inorganic spherulite under polarized filters demonstrating their size increase after mineralization. HRTEM image of apatite crystals showing their growth orientation and crystal lattices d-spacing. Surface characterization of the mineralized spherulite demonstrating; FIG. 7g-i SEM of a mineralized spherulite from the film surface at different length scales with nanocrystals growing in a spiral orientation around the center of the spherulite toward the surface of the film. FIG. 7j HR-TEM image from a FIB milling lift-out of a mineralized structure illustrating apatite crystals from the film surface. FIG. 7k SAED of the surface apatite crystals. Bulk characterisation of the mineralized film showing; FIG. 7l-n SEM image of the film cross-section from low to high magnification demonstrating the aligned apatite nanocrystals that are oriented parallel to each other. FIG. 7o HR-TEM image of apatite crystals showing their growth orientation and crystal lattices d-spacing. FIG. 7p SAED of the bulk apatite crystals. FIG. 7q FTIR spectra of mineralized film at 3 different time points; day 1, 3, and 7. FIG. 7r 19F solid-sate MAS-NMR spectra, FIG. 7s 31P MAS-NMR chemical shift, FIG. 7t Young's modulus and Hardness measurements of Keratin films before and after mineralization, where significance at p<0.05.

FIG. 8 shows the keratin mineralisation pattern over time. FIG. 8a is a polarized light microscopy of a keratin mineralized spherulite at day 1 of mineralization. FIG. 8b Light microscope image of spherulites under polarized light at day 1. The inset showing SEM of brushite-like crystals. FIG. 8c Keratin mineralized spherulite on day 2 of mineralization. d Polarized light microscope image of spherulites on day 2. FIG. 8e Coating of mineral on day 10 of mineralization displaying mineralized circular dis-ordered structures, which suggests that when the is no access to the keratin, mineralization is deposited on the top without displaying an ordered pattern.

FIG. 9 shows the crystal characterization of keratin induced mineralized structure. FIG. 9a shows High-resolution transmission electron microscopy (HR-TEM) images from a focused ion beam (FIB) milling lift-out of a mineralized structure illustrating three different zones; Top mineralized zone, middle interface zone, and bottom organic zone. The inset shows selected area electron diffraction (SAED) of the organic layer. FIGS. 9b and 9c show SAED of top and middle zones, respectively. FIG. 9d-h show HR-TEM of different areas on the FIB lamella numbered with respect to the numbers shown in FIG. 9a and demonstrating the crystal d-spacing that confirms the apatite phase. FIG. 9i is an energy dispersive x-ray analysis (EDX) of the FIB section displaying the chemical compound distribution in the three layers.

FIG. 10 shows Keratin film fabrication & characterization. FIGS. 10a and b show atomic force microscope (AFM) of keratin solution at 5 and 10 wt %. FIG. 10c shows SEM of a 3% keratin film. FIG. 10d shows fibril quantification of Keratin films. FIG. 10e shows 3 wt % keratin and triethylene glycol-dimethacrylate (TEGDMA) crosslinker (ratio of crosslinker to the lysine and cysteine amino-acid residues within the keratin of 1:10). FIGS. 10f and g show polarized light microscope images of spherulite organization patterns. FIG. 10h shows congo red staining of a keratin film demonstrating organic structures.

FIG. 11 shows the characterization of keratin in solution FIG. 11a-d show the self-assembly of the keratin films; FIG. 11a is 5 wt % keratin, FIG. 11b is 11 wt % keratin, FIG. 11c is 5 wt % keratin and triethylene glycol-dimethacrylate (TEGDMA) crosslinker (ratio of crosslinker to the lysine and cysteine amino-acid residues within the keratin of 1:11), FIG. 11d is 11 wt % keratin and triethylene glycol-dimethacrylate (TEGDMA) crosslinker (ratio of crosslinker to the lysine and cysteine amino-acid residues within the keratin of 1:11). FIG. 11e shows Keratin disorder-order retrieval; Attenuated total reflection-Fourier transform infrared (ATR-FTIR) deconvolution of the Amide I region for 2 different films 3 wt % keratin and 11 wt % keratin respectively, showing more β-sheet structures and less random coils when the keratin concentration increased, and their corresponding polarized light microscope images demonstrating a more ordered spherulites pattern when the keratin concentration is increased. FIG. 11f Fourier transform infrared (FTIR) imaging of an organic film demonstrating an area where a spherulite was imaged with light microscope. FTIR amide I (1600-1700 cm−1) and III (1200-1350 cm−1) spectra corresponding to four different areas of the film were outlined on the chemical map and captured and their corresponding secondary structure deconvolution percentages are presented in the relative pie charts.

FIG. 12 shows Organic-inorganic mechanism and mineralization tuneability. FIG. 12a shows Attenuated total reflection-Fourier transform infrared (ATR-FTIR) amide I deconvolution of the secondary structures in the organic keratin films that are FIG. 12a non-crosslinked or FIG. 12b crosslinked.

FIG. 13 shows inorganic spherulite characterization. FIG. 13a is Fourier transform infrared (FTIR) imaging of a mineralized film; the heat map on the left represents the distribution of the phosphate vibrations band region (900-1100 cm⁻¹), and amide I band region of the protein (1600-1700 cm⁻¹) are represented on the right, spectra were taken on four different regions of a mineralized keratin film according to their proximity to the protein/mineral, corresponding secondary structure deconvolution are presented in the relative pie charts. FIG. 13b is a SAXS scattering curve derived from an organic film showing a peak indicating a coil-like structure, FIG. 13c is a SAXS scattering curve derived from a non-mineralized film exhibiting a peak associated with a rod-like structure, FIG. 13d is a SAXS diffraction pattern of mineralized spherulites on a mineralized film, FIG. 13e shows scattering curves of each corresponding color in FIG. 13d.

FIG. 14 shows cell viability and RNA expression of human bone marrow-derived stem cells cultured on a membrane formed using one of (i) 5% keratin solution (Group 1); (ii) 5% keratin solution crosslinked with triethylene glycol dimethacrylate (TEGDMA) (Group 2); (iii) 5% keratin solution crosslinked with TEGDMA, followed by exposure to mineralisation solution (Group 3); (iv) 5% keratin solution crosslinked with hexamethylene diisocyanate (HDI) (Group 4) or Mem-Lok® Resorbable Collagen Matrix by BioHorizons. Cells were cultured for 7, 14, or 21 days before viability assessment. Cells were viable when cultured either in mesenchymal stem cell expansion media (a) or osteoblast growth medium (OGM) (b). Cells also showed messenger RNA expression after 28 days of culture, specifically of runt-related transcription factor 2 (RUNX2) (c), alkaline phosphatase (ALP) (d), osterix (OSX) (e), bone morphogenic protein 2 (BMP-2) (f), osteocalcin (OCN) (g) and osteopontin (OPN) (h). ** *** ** and * indicate significant differences of p<0.05, p<0.01, p<0.001 and p<0.0001 respectively.

FIG. 15 shows improvements in Knoop hardness and structural integrity after drop-cast treatment of white spot lesions (WSLs) with one of (i) 5% keratin solution (K₅); (ii) 5% keratin solution crosslinked with triethylene glycol dimethacrylate (TEGDMA) (K₅TE₁₀); or (iii) ICON® resin infiltrant (positive control) (ICON). Knoop hardness assessment was carried out in deionised water (DW) (a), artificial saliva (AS) (b) or mineralisation solution (MS) (c). Structural integrity improvements could be seen visually using scanning electron microscopy when comparing the surface view of the enamel surface of healthy enamel in DW (d), untreated WSLs in DW (e), WSLs treated with 5% keratin solution in DW (f) WSLs treated with 5% keratin solution in AS (g) and WSLs treated with 5% keratin solution in MS (h). Insert in (d) shows the border line separating the keratin treated and untreated WSL. ns, ** and **** indicate differences of p>0.05, p<0.01 and p<0.0001 respectively.

FIG. 16 is a diagrammatic representation of white spot lesion (WSL) artificial induction. (1) is 0.1 M lactic acid at pH 4.6, (2) is filter paper and (3) is 8% methylcellulose gel.

FIG. 17 is an artificially induced WSL sample in enamel.

FIG. 18 is an illustration of the pixel sampling method across the sample. (A) is the original sample image. (B) shows a dotted circle indicating the control baseline enamel part of the lesion. (C) shows a dotted circle indicating the intervention part of the lesion. (D) shows a dotted circle indicating the control WSL part of the lesion.

FIG. 19 is the optical coherence tomography measurement protocol. (A) shows the lesion WSL cross-sectional surface area traced with a dotted line (at the top of the images) and the grayscale value is this area is recorded. (B) shows the lesion area from A copied across to the intervention lesion. (C) shows the surface area of the lesion where the intervention was traced and recorded.

FIG. 20 shows white light microscopy (WLM) (left) and optical coherence tomography (right) both before treatment (lower section of the WLM image) and after treatment (upper section of the WLM image) with 5% of non-crosslinked keratin. Samples were in either deionized water (a) or artificial saliva (b).

FIG. 21 shows white light microscopy (WLM) (left) and optical coherence tomography (right) both before treatment (lower section of the WLM image) and after treatment (upper section of the WLM image) with 5% keratin crosslinked with tri-ethylene glycol dimethacrylate (TEGDMA). Samples were in either deionized water (a) or artificial saliva (b).

FIG. 22 shows white light microscopy (WLM) (left) and optical coherence tomography (right) both before treatment (lower section of the WLM image) and after treatment (upper section of the WLM image) with ICON® resin infiltrant (positive control). Samples were in either deionized water (a) or artificial saliva (b).

FIG. 23 shows grayscale levels (determined from optical coherence tomography) before and after treatment with ICON® resin infiltrant (positive control) in both deionized water (DW) and artificial saliva (AS). A high grayscale value indicates a greater severity of the WSL.

FIG. 24 shows grayscale levels (determined from optical coherence tomography) before and after treatment with 5% keratin without crosslinker in both deionized water (DW) and artificial saliva (AS). A high grayscale value indicates a greater severity of the WSL.

FIG. 25 shows grayscale levels (determined from optical coherence tomography) before and after treatment with 5% keratin with TEGDMA crosslinker in both deionized water (DW) and artificial saliva (AS). A high grayscale value indicates a greater severity of the WSL.

FIG. 26 shows grayscale levels (determined from optical coherence tomography) comparing controls against treatments of ICON® resin infiltrant, 5% keratin without crosslinker and 5% keratin with TEGDMA crosslinker in deionized water.

FIG. 27 shows grayscale levels (determined from optical coherence tomography) comparing controls against treatments of ICON® resin infiltrant, 5% keratin without crosslinker and 5% keratin with TEGDMA crosslinker in artificial saliva.

FIG. 28 shows data from a white spot lesion (WSL) pre-clinical study. FIGS. 28a and 28b Surface and cross-sectional views of the native polished enamel, respectively. FIGS. 28c and 28d Surface and cross-sectional views of the induced untreated WSL, respectively. FIG. 28e Interface between healthy and defective enamel showing the lost prism/inter-prism continua and pores created between prisms. FIG. 28f Image of a premolar tooth showing the treatment window on its facial surface; WSL on the right having chalky white characteristic appearance of the WSL and the keratin-treated lesion on the left with decreased opacity demonstrating the remineralization potential of the keratin. FIG. 28g Interface between WSL and lesion treated with keratin line of demarcation between the untreated and treated lesion, where the repaired layer could be noticed. FIG. 28h Surface view of the keratin-treated lesion showing the newly grown prismatic enamel that has filled the pores. FIG. 28i Cross section of g showing the newly formed enamel treated with keratin on the left compared to the lost prisms in the WSL on the right, FIG. 28j Enamel pores created due to inducing WSL; dotted arrows demonstrate the site of the pores and their relation to the c-axis. Keratin treated lesion incubated in mineralization solution displaying; FIG. 28k Enamel reconstructed with keratin filling up the pores. FIG. 28l High magnification SEM shows how the keratin has infiltrated the WSL in an organized pattern, some crystals having the same orientation of the prisms along the c-axis and others are oriented perpendicularly. FIG. 28m Repaired enamel from an area ~50 μm away from the enamel surface showing pores repaired with some areas appears to be filled with organic matrix. Keratin treated lesion incubated in artificial saliva demonstrating; FIG. 28n Surface view displaying the border between the treated and untreated WSL, the inset shows the repaired enamel with keratin with almost no gaps, FIG. 28o Border line separating the repaired enamel (top) from the lesion gaps (bottom) at a depth ~ 100 μm away from the surface, FIG. 28p High magnification of the repaired enamel treated with keratin exhibiting elongated crystals, filling the lesion gaps, and their direction parallel to each other. FIGS. 28q and 28r High-resolution transmission electron microscopy (HR-TEM) of focused ion beam (FIB) lamella showing continuous non-porous enamel. FIG. 28s selected area electron diffraction (SAED) of the repaired region. FIG. 28t Microhardness analysis. FIG. 28u Nanoindentation measurements showing elastic modulus and hardness, where significance at p<0.0001. P: prism, IP: inter-prism, UPW: ultra-pure water, MS: mineralization solution, AS: artificial saliva.

FIG. 29 shows surface characterization of treated enamel lesions. FIG. 29a Integration between the keratin and the newly-formed enamel-like crystals demonstrating their attachment to the organic matrix which seems to be guiding their growth. WSL treated with keratin incubated in artificial saliva demonstrating; FIG. 29b keratin coating in ultra-pure water (UPW). FIG. 29c Mineralization pattern of untreated WSL in mineralization solution. FIG. 29d-f Resin treated lesions incubated in mineralization solution showing resin coating with mineralized precipitates on top.

FIG. 30 shows focused ion beam (FIB) lift out and mechanical characterization. FIGS. 30a and 30b High-resolution transmission electron microscopy (HR-TEM) of WSL treated with keratin. FIG. 30c selected area electron diffraction (SAED) diffraction pattern of lesion treated with keratin 50 μm away from the surface. FIG. 30d Bulk nanoindentation results of the WSL before and after treatment, where significance at p<0.0001.

FIG. 31 shows in vivo rat calvarial defect regeneration. FIG. 31a is an illustration demonstrating the surgical procedure performed. FIG. 31b shows the rat calvarial defect before bone removal. FIG. 31c shows the rat calvarial defect after bone removal. FIG. 31d shows the rat calvarial defect after applying keratin film. FIG. 31e-p show X-ray with scale bar 6 mm (31e-31j) and Micro-CT 3D construction images (31k-31p) of empty defect (31e, 31k), Ker5 (31f, 31l), Ker5TE1 (31g, 31m), M-Ker5TE1 (31h, 31n), Ker5H3 (31i, 31o), and collagen membrane (31j, 31p). FIG. 31q-u shows measured parameters by Micro-CT; bone volume (BV) (mm2) (31q), ratio of bone volume to trabecular volume (BV/TV) (%) (31r), trabecular thickness (Tb·Th) (mm) (31s), trabecular number (T·N) (mm−1) (31t), and trabecular separation (mm) 31(u).

### DETAILED DESCRIPTION

### Examples

### Example 1—Keratin Extraction, Keratin 75 Production and In Vitro Membrane Formation Keratin Extraction

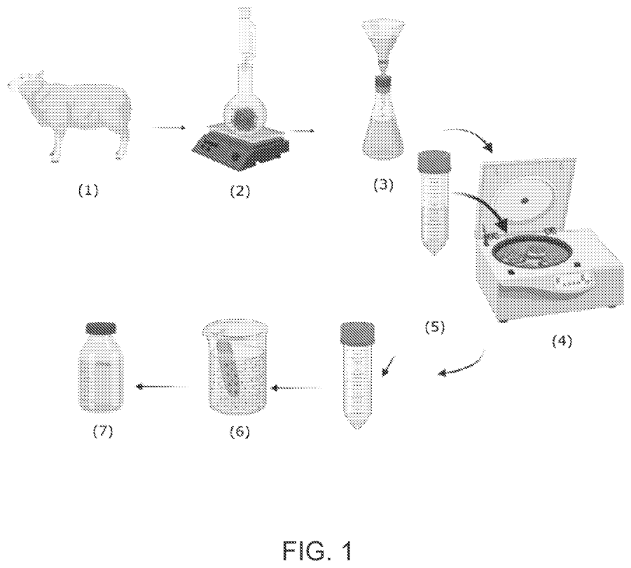

The extraction process is set out in FIG. 1. Sheep's wool was thoroughly rinsed with deionised water and dried. As wool (as well as other keratin sources such as hair) is coated in a thin covalently bonded lipid layer, the wool was defatted by Soxhlet extraction for 6 hours using hexane and dichloromethane 1:1 v/v for refluxing. This allows for better extraction of the keratin. Cleaned wool (10 g) was mixed with 7M urea (180 mL), Sodium Dodecyl Sulfate (SDS) (0.021 mol, 6 g) and 2-mercaptoethanol (0.21 mol, 15 mL) in a 300 mL round-bottom flask. The mixture was heated at 60° C. for 24 hours with continuous stirring and maintained in a neutral pH range. The 2-mercaptoethanol aids keratin solubilisation by breaking the disulphide bonds between cysteine residues in the amino acid chain. Raising the temperature removes the protons from the —NH₃ groups, breaking the hydrogen bonds. Urea was used at high concentrations in order to swell the keratin fibre through weakening the hydrophobic interaction between keratin fibres, which facilitates the reduction of disulfide bonds by 2-mercaptoethanol. This in turn allows for increased diffusion rates of the reducing buffer into the fibres and proteins out of the fibres. SDS was employed to break the strong intermolecular interactions and speed up the extraction rate, as well as contribute to the aqueous solution's stability.

The resultant mixture was filtered through a 120 stainless-steel mesh sieve and then centrifuged for 30 minutes at 6,000 rpm. The supernatant was subsequently dialyzed (5 kDa cut off) against deionized water (3.5 L) for 3 days, until a colourless clear solution was obtained with regular changing of the outer water (two to three times). Aliquots of the reduced keratin solution were kept in a freezer at −80° C. for 4 hours and then freeze-dried (VirTis SP Scientific Sentry 2.0, Ipswich, UK) until a fine lyophilized white interwoven fibrous powder remained.

Keratin 75 expression in Escherichia coli and Purification

Keratin 75 (KR75) was produced in Rosetta BL21 E. coli cells (Novagen) using a plasmids pET-15b (+) expression system adjusted with an HRV 3C protease site (Synbio Technologies, LLC) and developed with 6 Histidine tags. The inoculum was prepared by cultivation of the selected transformant in LB broth containing 100 mg·ml⁻¹ ampicillin, the culture was then incubated at 37° C. for 4 hours, achieving an OD600 nm~0.6). Recombinant protein expression was induced by adding isopropyl β-D-1-thiogalactopyranoside (IPTG) at 18° C. overnight in a shaker. The cells were harvested by centrifugation at 6000 g for 30 minutes, re-suspended in 0.05 M Tris buffer (pH 6.0) and were then lysed by sonication. The sonicated mixture was centrifuged at 7000 rpm for 30 minutes. Expressed KRT75 cell pellets were washed 4 times with wash buffer pH 7.5 containing 0.05 M Tris-CI, 5 mM EDTA and 0.5% Triton-X100 to remove the cell debris.

Afterwards, the cell pellets were solubilized in 1 M urea, 2 mM DTT in 0.05M tris buffer pH 12. The produced mixture was then dialyzed against 0.05 M tris buffer pH 7.5 using (12-14 kDa cutoff) dialysis bag. Purification for the isolated protein was done by using HisPur™ Ni-NTA Spin Columns (Thermo Scientific™). Dialysis was them done against Milli-Q water pH 7.0 for the to remove the imidazole and was then freeze-dryer (VirTis, SP scientific-sentry 2.0)−60° C. for to get the KRT75 powder.

### In Vitro Membrane Formation

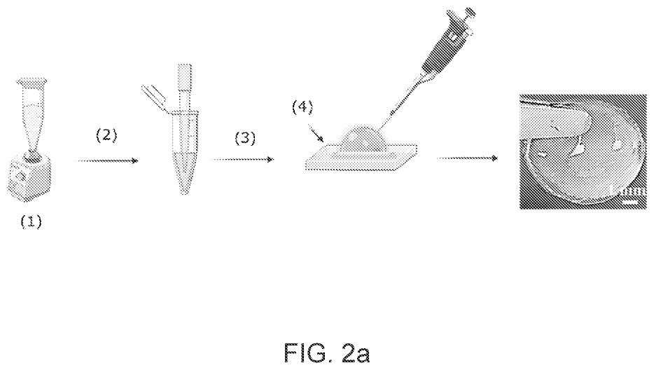

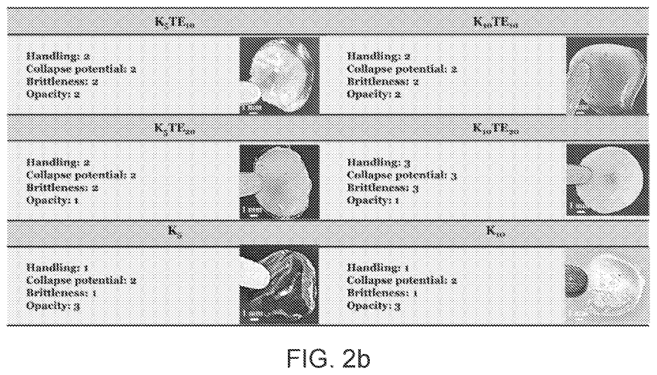

After keratin extraction or production, freeze-dried keratin powder was dissolved at concentrations ranging from 2-10 wt % in one of Milli-Q water pH 7-11, dimethyl sulfoxide (DMSO), or Dimethylformamide (DMF). The resultant solution was either non-crosslinked or cross-linked using either tri-ethylene glycol dimethacrylate (TEGDMA), hexamethylene diisocyanate (HDI) or poly-ethylene glycol dimethacrylate (PEGDMA) (with the addition of 3% wt lithium phenyl-2,4,6-trimethylbenzoylphosphinate (LAP) photo-initiator). The solution is then drop casted on a polydimethylsiloxane (PDMS) substrate and left to dry at one of ambient room temperature, 37° C. or 45° C. to induce self-assembly (FIG. 2a).

FIG. 2b shows the variety of membranes that be formed when parameters such as keratin concentration of the starting keratin solution, use of crosslinker and concentration of crosslinker are varied. Membranes formed with a crosslinker took 10 to 15 minutes to dry, whilst membranes formed without a crosslinker took 15 to 20 minutes to dry.

### Example 2—Keratin Variation Analysis

### Methodology

To determine the molecular weight of the keratin structures after self-assembly, samples were analysed using sodium dodecyl sulphate-polyacrylamide gel electrophoresis (SDS-PAGE). Each aliquot of the LDS sample buffer (10 μL) was combined with 20 μL of all soluble fractions and a 1 mg/ml powder dilution before being heated at 70° C. for 10 minutes. Then, 10 wells of 4-12% Bis-Tris NuPAGE® precast polyacrylamide gel (Thermo Fisher Scientific, UK) with ethane sulfonic acid (MES buffer) were loaded with 20 μL of each solution. At 200 V, 125 mA, electrophoresis was carried out for 40 minutes. The gels were then stained for 25 minutes with two parts Coomassie Brilliant Blue, three parts 10% acetic acid, and overnight with 10% acetic acid and deionized water.

Further confirmatory analysis was done to validate the previous SDS_PAGE findings. The band from the purified protein SDS-PAGE was sent for MS analysis to confirm the expression of KRT75 protein. The gel band was prepared for enzymatic digestions. Cys residue was reduced with DTT and derivatised by the aid of iodoacetamide to form constant carbamidomethyl derivatives. Trypsin digestion was conducted overnight at RT after primary incubation at 37° C. for 2 hours. Raw data of mass spectrometer was inserted into the peak list folder by Proteome Discoverer (ThermoScientific; v2.5). The data file was processed and searched using the Sequest (Eng et al; PMID 24226387) search algorithms against the Uniprot Human Taxonomy database (50,590 entries). Database searching was performed at a stringency of 1% FDR including a decoy search. The database output file was uploaded into Scaffold software (version 5.1.2; www.proteomesoftware.com) for visualisation and manual verification.

### Results

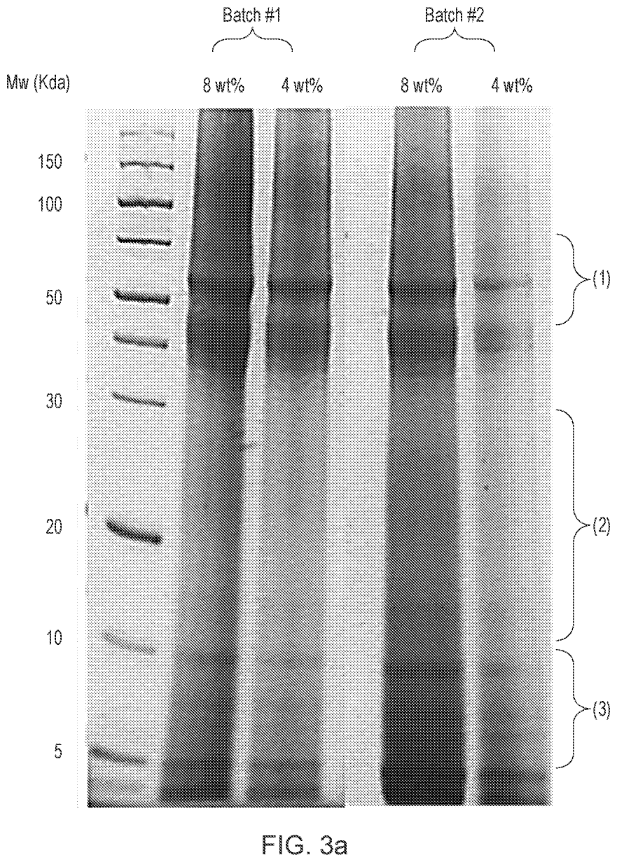

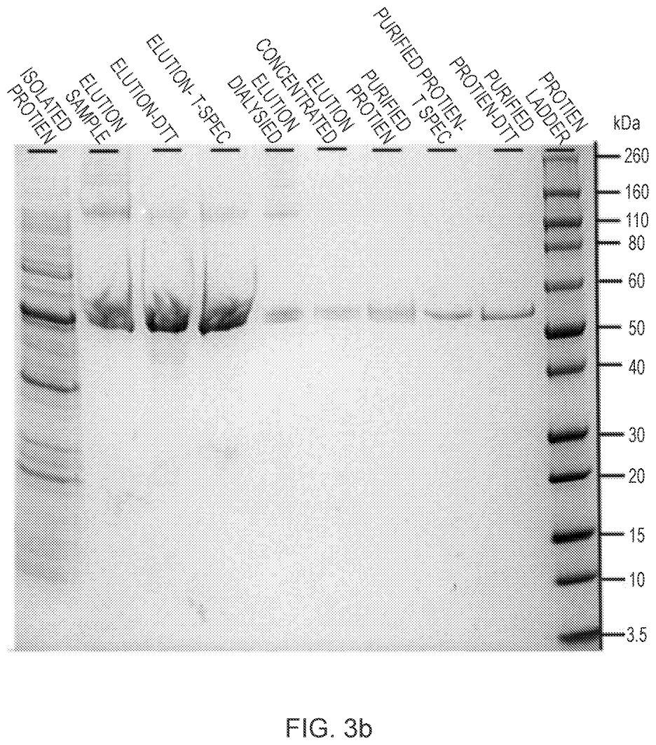

FIG. 3a shows that keratin derived from sheep wool using the method as set out in Example 1 results in a high variety of keratins with varying molecular weight. The Figure shows clear band at 45-50 kilo Daltons (kDa) relating to type I keratin, and a clear band at 55-60 kDa relating to type II keratin. These low-sulphur (LS) keratins form intermediate filaments in the wool fibre cortex. A series of high sulpur (HSPs) keratins (at 12 to 28 kDa) and high glycine-tyrosine (HFT) keratins (at less than 10 kDa) could also be seen and these keratins are derived from the wool matrix.

By contrast the artificially expressed keratin 75 expectedly resulted in keratin of a single molecular weight (FIG. 3b).

### Example 3—Imaging of Keratin Structures and Mineralisation

### Methodology

To induce protein-mediated mineralization in the membranes, a super saturated calcium-phosphate mineralization solution was prepared by adding 2 mM Hydroxyapatite (HAP) powder and 2 mM of sodium fluoride to 100 ml of deionized water with continuous stirring. To completely dissolve the powder, 69% nitric acid was added dropwise into the solution slowly until the powder was completely dissolved. Ammonium hydroxide solution (30%) was added dropwise until the pH reached 6. The cross-linked keratin membranes were incubated in 50 mL of the HAP solution at 37° C. for 3 to 5 days. After mineralization, the membranes were removed from the solution, thoroughly rinsed with deionized water, and stored at room temperature until characterization.

The morphology of the self-assembled keratin fibrils was analysed by Scanning Electron Microscopy (SEM). Keratin membranes were mounted on aluminium stubs with double-sided carbon tape. Samples were sputtered with 8 nm conductive gold coating (Leica EM ACE600 sputter coater, Milton Keynes, UK). SEM images were obtained using a Schottky field-emission JSM-7610F SEM (JEOL Ltd., Hertfordshire UK), with the surface topography being observed using the secondary electron detector.

Membranes were observed by cross-polarized light microscopy (GXM-XPLPOLTEC-5, UK) with an air objective 4 and 10× in which the polarizer and analyser were fixed perpendicularly to each other.

### Results

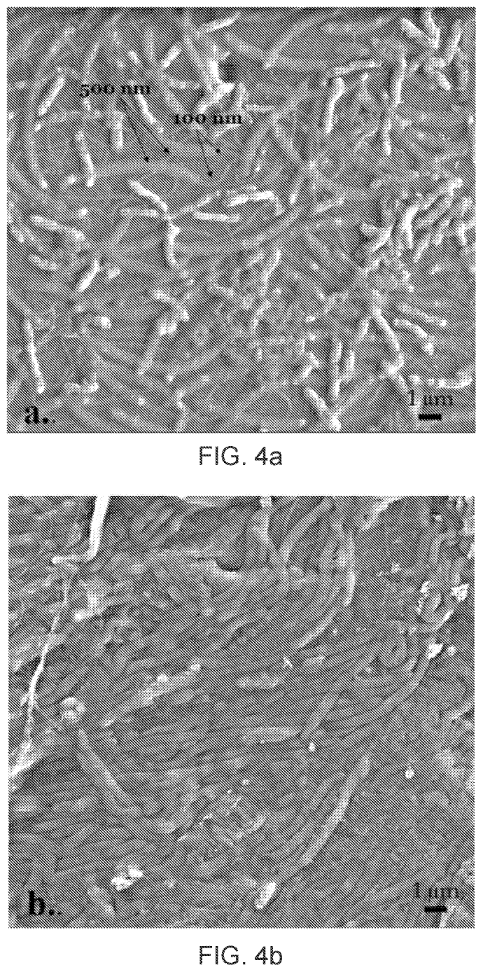

For some membranes, the resultant solution was cross-linked using triethylene glycol-dimethacrylate (TEGDMA) in different molar ratios to the cysteine and lysine residues present in the keratin to control the stiffness/co-assembly of the membranes. These crosslinked membranes were able to self-assemble into nano fibres ranging from 100-500 nm in diameter, and they were able to self-assemble in an organized manner when the keratin concentration was increased from 5 wt % (FIG. 4a) to 10 wt % (FIG. 4b) while all other conditions were similarly maintained. Increasing the keratin concentration showed fibres of more organized self-assembly appearing to gather in bundles.

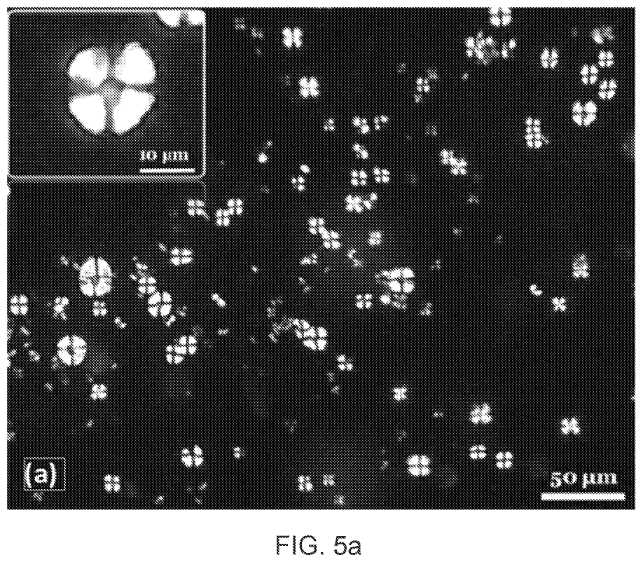

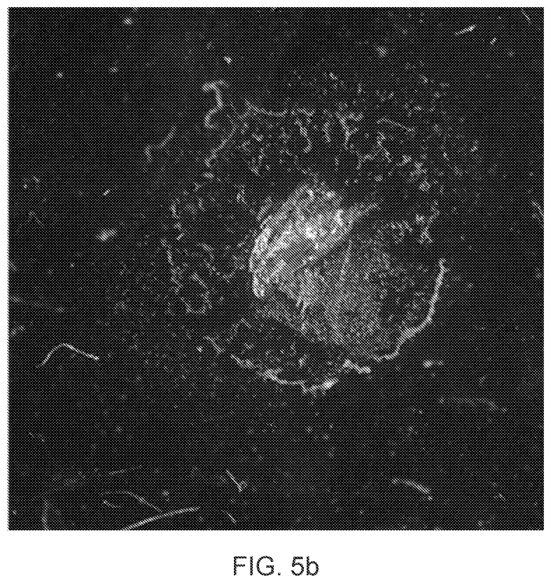

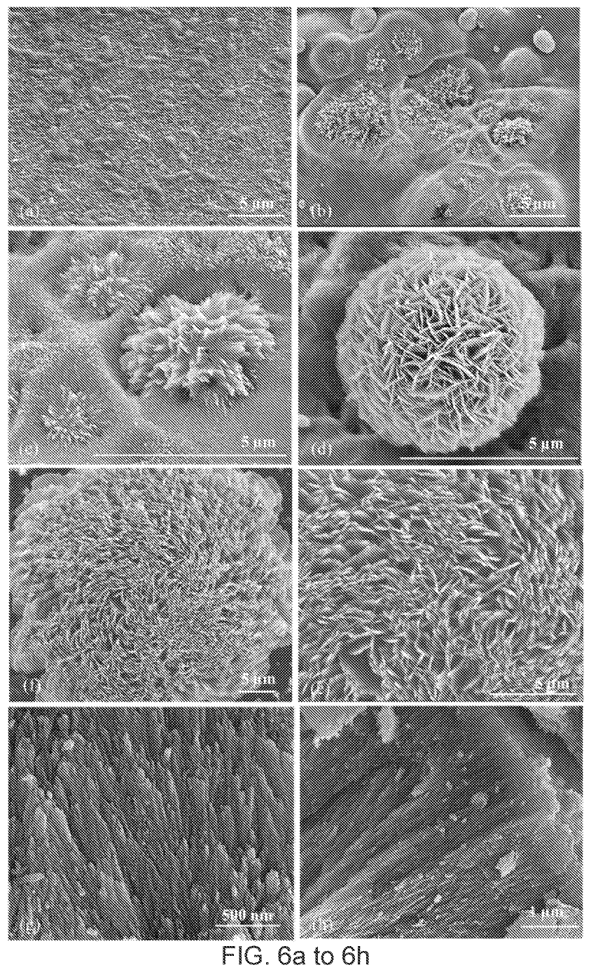

Polarised light microscopy revealed the formation of spherulite structures (which appear as a Maltese cross in shape) made of wool-derived keratin (FIG. 5). Spherulites are anisometric polycrystalline structures formed by the self-assembly of the crystalline lamellae into spherical superstructures, in which the lamellar sheets grow out radially from the centre. Spherulites are seen at 5% wt and 10 wt % keratin, and both with and without crosslinker. By contrast, no spherulites were seen when equivalent membranes were made from keratin 75.

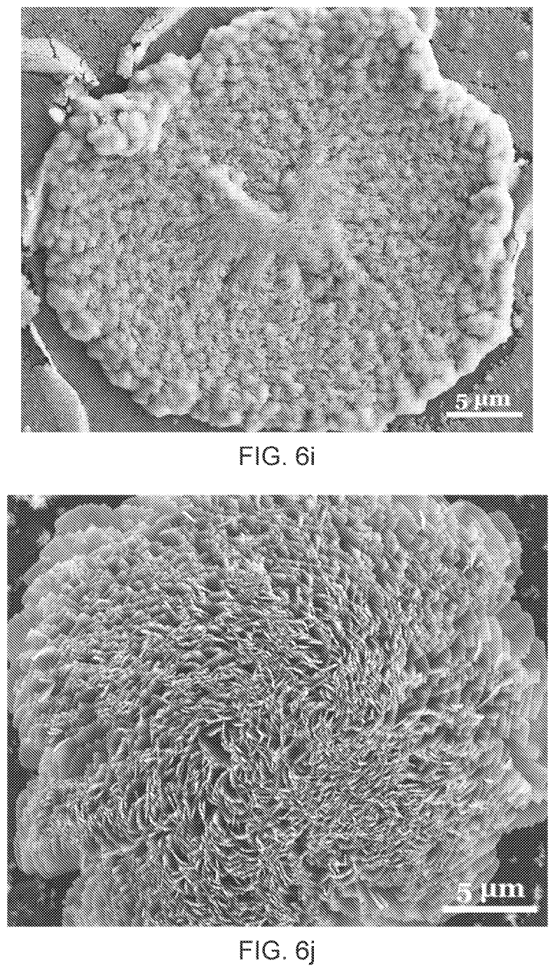

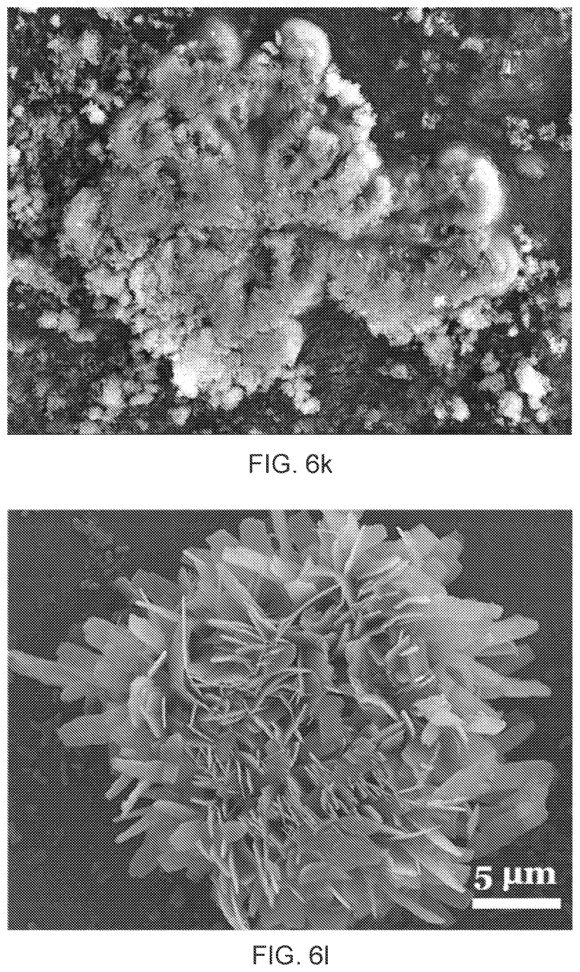

FIGS. 6a to 6h show how mineralisation takes place and how spherulites can act as nucleators to mediate an ordered mineralisation pattern.

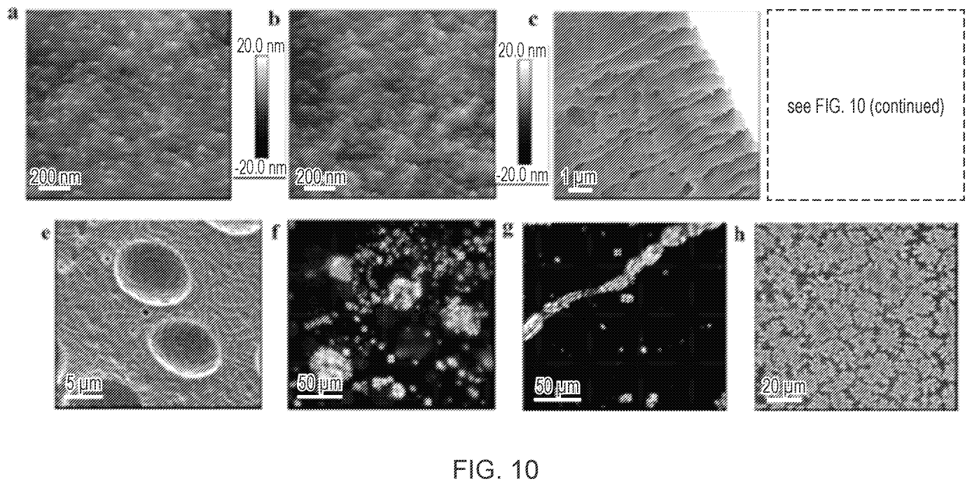

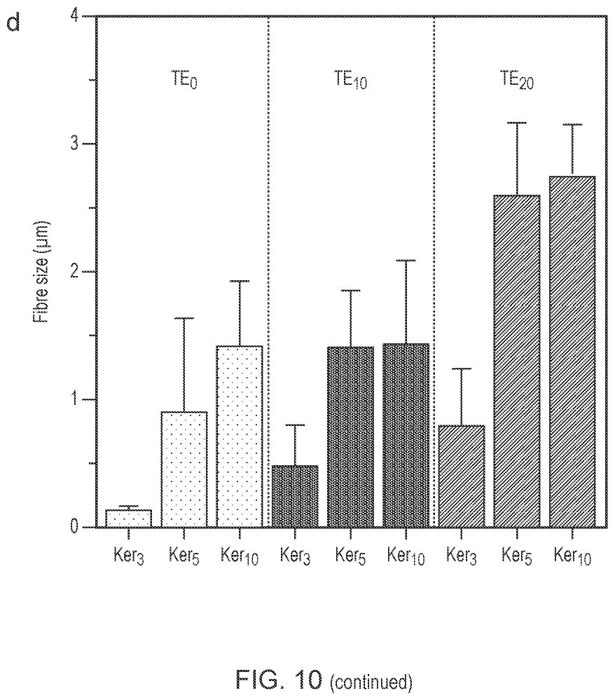

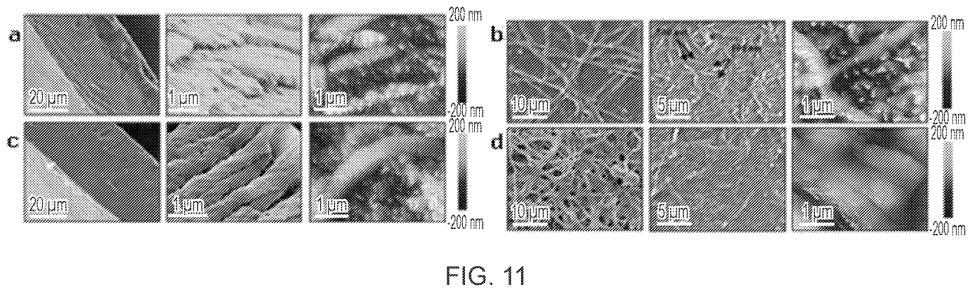

In vitro mineralisation led to the production of nanocrystals, proving that the keratin nanofibres acted as nucleators for biomimetic mineralization at a keratin concentration of 5% (FIG. 6i) and 10% (FIG. 6j) with triethylene glycol-dimethacrylate (TEGDMA) crosslinker.

FIG. 6k shows mineralisation of keratin crosslinked with poly-ethylene glycol dimethacrylate (PEGDMA) after the addition of 3% wt lithium phenyl-2,4,6-trimethylbenzoylphosphinate (LAP) photo-initiator. FIG. 6l shows mineralisation of a keratin membrane formed without crosslinker. It is believed that the spherulites act as the nucleation points.

### Example 4—Mineral Nucleation and Crystal Growth in Keratin Films

Natural keratins were extracted and the extracted powder was solubilized in ultra-pure water (UPW) at 3%, 5% and 10% w/v. The films were then allowed to air dry in order to allow the self-crosslinking of the cysteine residues, and reconstitute the disulfide bonds, which renders the films to become insoluble.

Introducing functional groups into the protein backbone can enhance the reactivity and the supra molecular properties. Therefore, in addition to the keratin only group, films were also processed by mixing the solubilized keratin using Tri-ethylene glycol di methacrylate (TEGDMA).

The organic matrix of the keratin-based films was investigated.

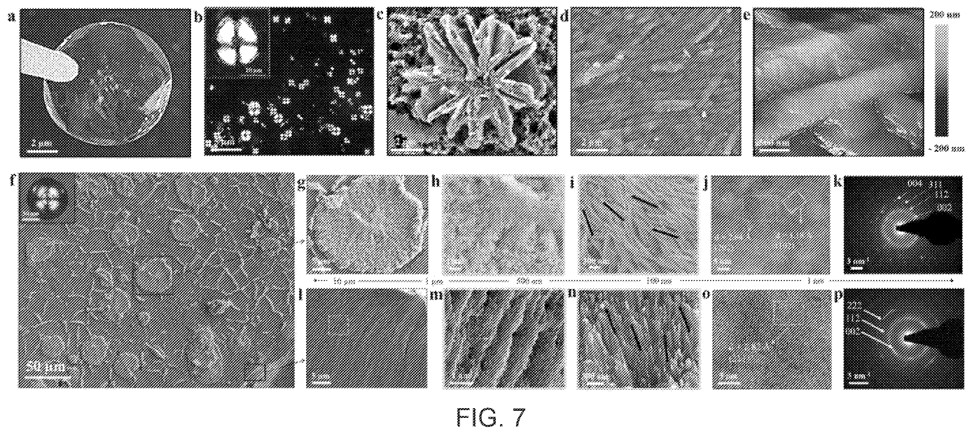

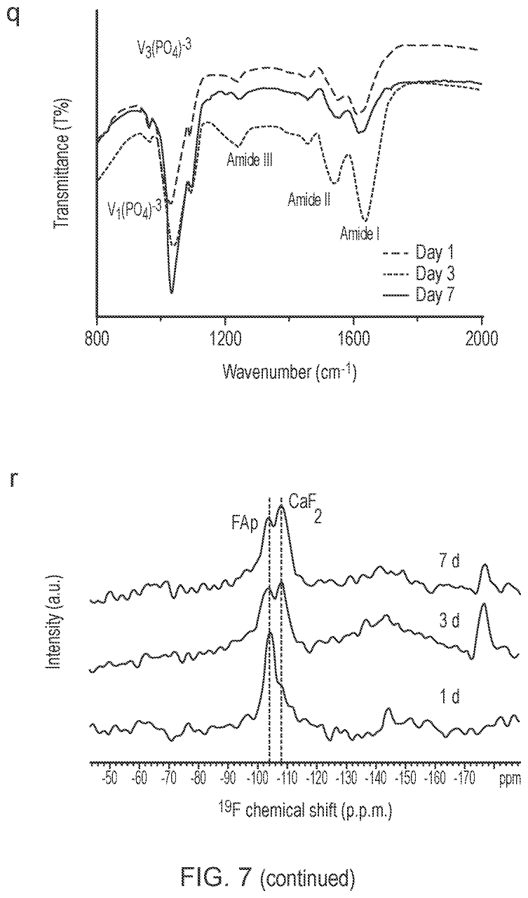

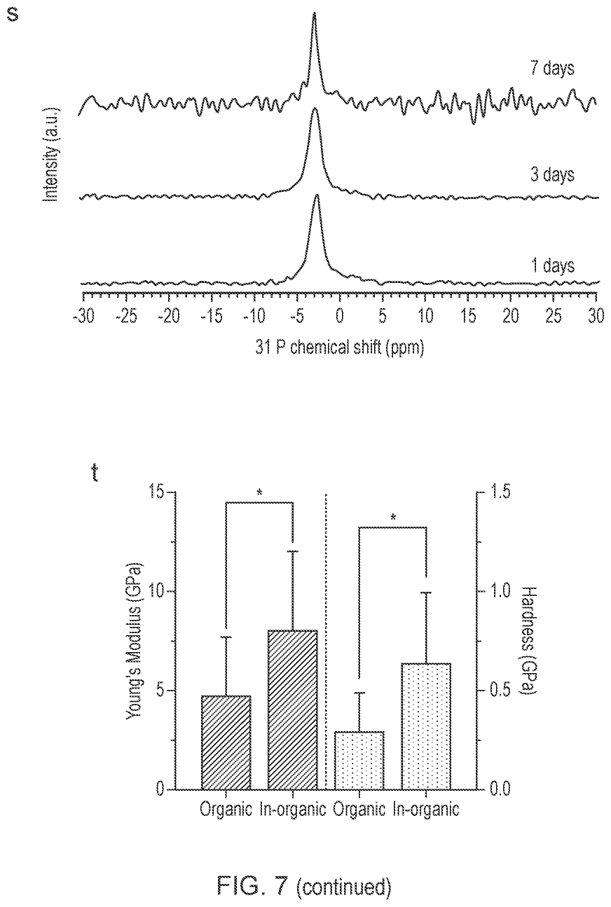

FIG. 7a shows a keratin film before mineralization.

FIGS. 7b and 7c show the network of birefringent organic spherulites displayed by all keratin-based films and FIGS. 7d and 7e show the microfibrillar structures within their matrix. The spherulites are thought as crystalline aggregates of closely packed chains that can provide the protein with significant strength and rigidity. These spherulites diameter ranges from (1-10 μm) and are characterized by the Maltese cross appearance when viewed between polarizing filters.

The mineralization potential of these films was then established after incubating them in a supersaturated fluoride-rich hydroxyapatite solution at 37° C. The mineralization pattern was tracked over time using scanning electron microscopy (SEM).

Samples were mounted on aluminum stubs via carbon-adhesive tape and were coated with 10 nm thick gold coating using an auto sputter coating machine coating (Leica EM ACE600 sputter coater, Milton Keynes, UK). SEM images were obtained using a Schottky field-emission JSM-7610F SEM (JEOL Ltd., Hertfordshire UK) operated at 10 kV, with the surface topography being observed using the secondary electron detector. Images were then transferred to ImageJ (NIH, US) for quantitative analysis. Induced enamel lesions were prepared by etching with phosphoric acid 35% for 20 seconds and rinsed for 20 seconds for removing the smear layer.

FIG. 7f shows the spherulite-like mineralized structures detected under SEM.

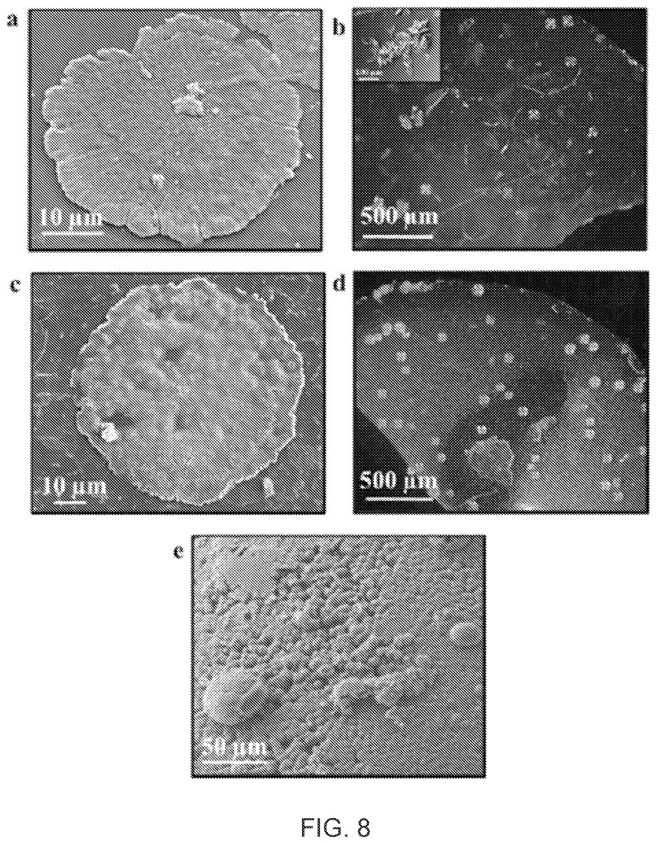

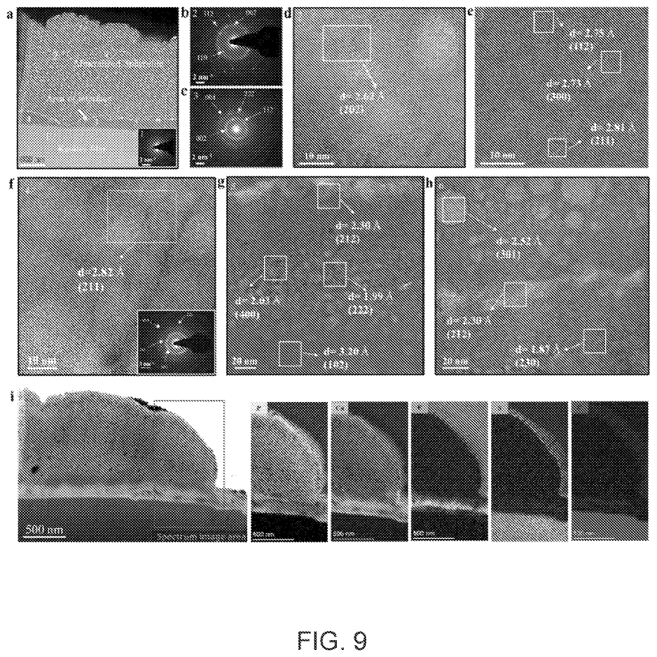

FIGS. 8a and b show that on the first day after mineralization, a smaller number of spherulites are present with some platelet-like crystals.

FIGS. 8c and 8d and show that on the 2nd day, some apatite crystals started to evolve, and the platelet-like crystals started to disappear with the spherulites increasing in number.

FIG. 7g-i show that on day 3, the mineralized spherulitic structures started to demonstrate spirally oriented morphology of assembled mineral nanocrystals needle-like in shape and appeared to be composed of aligned prism-like structures of 1.36±0.33 μm thick and tens of micrometers in length. These prisms are made of elongated nanocrystals, their average diameter is 83±22.96 nm, their provenance is the center of the spherulite and terminate on a point at the spherulite periphery.

FIG. 7l-n show that within the bulk of the mineralized structures, the prisms comprised of elongated apatite nanocrystals and were aligned in a more organized and parallel arrangement.

High-resolution transmission electron microscopy was performed. The FIB prepared lamellas were characterized using a Thermo Fisher 60-300 kV Spectra Ultra TEM equipped with an Ultra-X EDS detector and Cs aberration corrector. Images were acquired on a 4k×4k Ceta-S detector. Data was post processed using Velox® software, version 3.8. The obtained images were analyzed using the Gatan Microscopy Suite® (GMS 3) software. For the analysis of crystal phases present in the samples, d-values obtained from SAED patterns were compared against PDF2 database (ICDD, USA, release 2009).

FIGS. 7j and 7o confirm the apatitic phase by high-resolution transmission electron microscopy (HR-TEM) and selected area electron diffraction (SAED) and crystallite d-spacings data analyses further confirm the presence of apatite crystals on both the surface and cross-section of the mineralized spherulite.

FIG. 7k is a SAED data analysis displaying a polycrystalline pattern corresponding to apatite on the film surface.

FIG. 7p shows the crystalline pattern showed arching at 002 while in the bulk. This arching could be attributed to the texture and preferential orientation of the crystals within the bulk of the keratin.

### Example 5—Chemical and Mechanical Characterization of Mineralized Films

Fourier transform infrared (FTIR) analyses were carried out at three different time points of mineralization day 1, 3, and 7 to investigate the mineral compounds in the films.

FTIR analysis was conducted on the keratin solutions and films using Spectrum One FTIR Spectrometer (PerkinElmer®, Buckinghamshire, UK) in ATR mode, using a white light source and an InGaAs detector. Spectra were taken between wavenumber 4000 to 700 cm⁻¹ by averaging 32 scans per sample at a resolution of 2 cm⁻¹. Amide I spectral region (1700-1600 cm−1) was analyzed to compare the keratin secondary structure composition using OriginPro 8.5 software (Microcal Inc.). The assignments of spectral bands were as follows: 1610-1627 cm⁻¹ intermolecular β-sheets, 1628-1642 cm⁻¹ β-sheets, 1643-1650 cm⁻¹, 1643-1650 cm⁻¹ random coils, 1650-1659 cm⁻¹ α-helix, 1643-1650 cm⁻¹, and 1660-1699 cm⁻¹ β-sheets/β-turns.

Magic angle spin-nuclear magnetic resonance was also performed. Solid-state 19F MAS-NMR analysis was conducted using a 14.1 Tesla spectrometer (600 MHz, Bruker, Coventry, UK) at a Larmor frequency of 564.5 MHz under spinning conditions of 22 kHz in a 2.5 mm rotor to investigate the fluoride and phosphorous compounds in the mineralized films. All samples were crushed into fine powder using gyro mill machine (Gyro mill, Glen Cresto, London, UK), and then analyzed. The spectra were acquired from a single-pulse experiment of 60 s recycle duration. The 19F chemical shift scale was calibrated using the—120 p.p.m. peak of 1M of NaF solution along with trichloro-fluoro-methane (CFCl3), as a second reference. H3PO4 was the reference material for the chemical shift in 31P. Spectra were acquired for 4 h with accumulation of 240 scans.

FIG. 7q shows phosphates (PO₄³⁻) bands that correspond to the typical apatite structures were detected at the three time points. The IR analysis detected intense peaks that are attributed to the PO₄³⁻ v3′ and v3′″ anti-symmetric stretching vibrational modes of phosphate bands between 1000-1100 cm⁻¹. PO₄³⁻ v1 symmetric stretching vibrational mode were also observed between 960-965 cm⁻¹.

FIGS. 7r and 7s verify the crystalline phase using Magic angle spin-nuclear magnetic resonance (MAS-NMR). F MAS-NMR spectra of the films at day 1, demonstrated only a sharp peak at around −103 ppm, which corresponds to fluorapatite (FAp). A fluorite (CaF₂) peak was observed at −108 ppm on day 3 along the FAp and was more pronounced at day 7 (FIG. 7r). ³¹P MAS-NMR spectra of the keratin films at the different time points (FIG. 7s) revealed a sharp peak at ~3 ppm which is assigned to crystalline apatite.

Young's modulus and hardness measurements were investigated. Keratin films and enamel samples were glued onto an aluminum holder. Nanoindentation tests were carried out by nanoindenter (iNano) by Nanomechanics, Inc. (maximum indentation load of 50 mN). The mechanical properties of the mineralized films were recorded with an indentation depth of 30 nm on the films in order to decrease the organic tissue influence underneath. Enamel samples Young's modulus and hardness were evaluated following a previously established method45 with a maximum depth of 1000 nm with 50-100 indents were made for each sample.

FIG. 7t shows Young's modulus and hardness of Kertain 5 wt %, tested before and after mineralization. Kers exhibited modulus (5.11±3.0 GPa) and hardness (0.3±0.2 GPa) before mineralization, while the films after mineralization demonstrated an increase in both stiffness and hardness where Elastic modulus=8.1±3.94 GPa, and Hardness=0.64±0.35 GPa.

### Example 6—Crystallographic Characterization of the Mineralized Structures to Further Investigate the Preferential Growth and Crystallographic Diffraction of the Nanocrystals, Series of Ultrathin Sections of Mineralized Spherulites were Milled Via Focused Ion Beam (FIB) and the FIB Prepared Lamella were Investigated Using High-Resolution Transmission Electron Microscopy (HR-TEM) and Selected Area Electron Diffraction (SAED)

Focussed ion beam-SEM was performed as follows. FIB milling and deposition were performed using a Thermo Scientific™ Helios™ 5 UC DualBeam or Zeiss Crossbeam 350 FIB-SEM at Ga ion beam parameters, 30 kV and 1 nA, except for a final low-voltage cleaning step done with a 2 kV beam to provide additional gentle thinning while reducing Ga implantation and amorphization damage. The region of interest (ROI), approximately 20×20 μm2-wide, was coated with a thin layer of carbon and thicker, ~2 μm, tungsten layer by electron and ion beam deposition. The specimen was extracted with a micromanipulator and attached to a Cu-grid. Sample pores were filled with tungsten deposition to improve the structural integrity of the sample during thinning. The lamella was thinned with three or four windows, ~3 μm wide, depending on the size of the mineral specimen chosen. Electron beam deposition was done at a stage tilt of 0°, ion beam deposition and trench milling were done at 52° (plus a small over-tilt angle of ~0.7° to obtain parallel front and back faces for thinning), and lift-out and attachment were done at 0°.

High-resolution transmission electron microscopy was performed as follows. The FIB prepared lamellas were characterized using a Thermo Fisher 60-300 kV Spectra Ultra TEM equipped with an Ultra-X EDS detector and Cs aberration corrector. Images were acquired on a 4k×4k Ceta-S detector. Data was post processed using Velox® software, version 3.8. The obtained images were analyzed using the Gatan Microscopy Suite® (GMS 3) software.

For the analysis of crystal phases present in the samples, d-values obtained from SAED patterns were compared against PDF2 database (ICDD, USA, release 2009).

FIG. 9a-9h show that the mineralized spherulite constitutes three different areas: a Bottom layer, which was completely made up from the keratin organic matrix and no crystal diffraction pattern was observed; a top layer, displaying several circular structures on TEM of an average diameter 22.55±4.36 nm (FIG. 9a, d), these structures start to decrease in number and become smaller in size top to bottom, they exhibit a polycrystalline pattern on SAED (FIG. 9b); and an interface layer (FIG. 9e-h) that lies between the other two layers where the circular structures started to almost disappear, their SAED array demonstrated crystalline rings in the form of arches. D-spacing analysis confirms the diffraction patterns which match those of hydroxyapatite and fluorapatite crystals.

FIG. 9i is an energy dispersive x-ray analysis (EDX) showing that the top layer is made up of large amounts of calcium and phosphorous and smaller amounts of sulfur, carbon, and fluoride, while the interface constitutes mainly fluoride, and the bottom is made up entirely of carbon and sulfur.

### Example 7—Organic Matrix Surface Morphology and Self-Assembly and Keratin Structural tuneability

To investigate the role keratin organic matrix assembly and conformation plays in the mineralization process mechanism, keratin secondary structures were investigated using circular dichroism (CD) and dynamic light scattering (DLS) in aqueous solutions at different pH.

Secondary structure of keratin in aqueous solution of different pH were investigated by CD spectroscopy (Chirascan™ CD Spectrometer, Applied Photophysic Limited, UK) equipped with a temperature controller, the final pH for both solutions was stable over time. The keratin solutions (0.2 mg/ml) were prepared in Milli-Q water. And to test the effect of Calcium ions on the keratin secondary structure, lyophilized keratin (0.2 mg/ml) was dissolved in 5 mM Calcium Chloride (CaCl2). A quartz cuvette with 0.5 mm path length was used for the measurements and CD spectra were obtained by signal integration 3 scans from 190 to 260 nm at a scan rate of 50 nm/min with a bandwidth of 1 nm. The spectra were acquired at 25° C. The solution was equilibrated for 5 min before scanning. CD data sets were then deconvoluted using Dichroweb web server for the calculation of protein secondary structures.

In order to optimize the formation of the keratin films, DLS was performed to measure changes in the particle size of keratin in solution and comparing their charges. Zetasizer (Nano-ZS ZEN 3600, Malvern Instruments, UK) was used for measuring both the Z-potential and Z-average measurements. The keratin solutions (0.2 mg/ml) were prepared in Milli-Q water of different pH at 25° C. Also, to test the interaction of keratin with Calcium salts, lyophilized keratin (0.2 mg/ml) was dissolved in 5 mM CaCl2. Each sample was equilibrated for 5 min before measurements.

Atomic Force Microscopy (AFM) was also performed. Keratin films with or without mineralization were imaged in tapping mode under ambient conditions using a MultiMode® AFM with a Nanoscope III controller (Digital Instruments, Santa Barbara) and OTESPA-R3® cantilevers (Bruker, California). Image analysis was performed using NanoScope® analysis (Bruker, California) where the images were flattened to remove curvature and slope.

FIGS. 10a and b show, upon solubilizing the keratin in water, a number of nanospheres whose diameter ranges between 20-50 nm with a mean diameter of 33.02±6.87 nm are observed using atomic force microscope (AFM) in solution.

FIG. 10c shows an organic network of nanospheres packed on the surface of a 3 wt % keratin solution that has been allowed to dry.

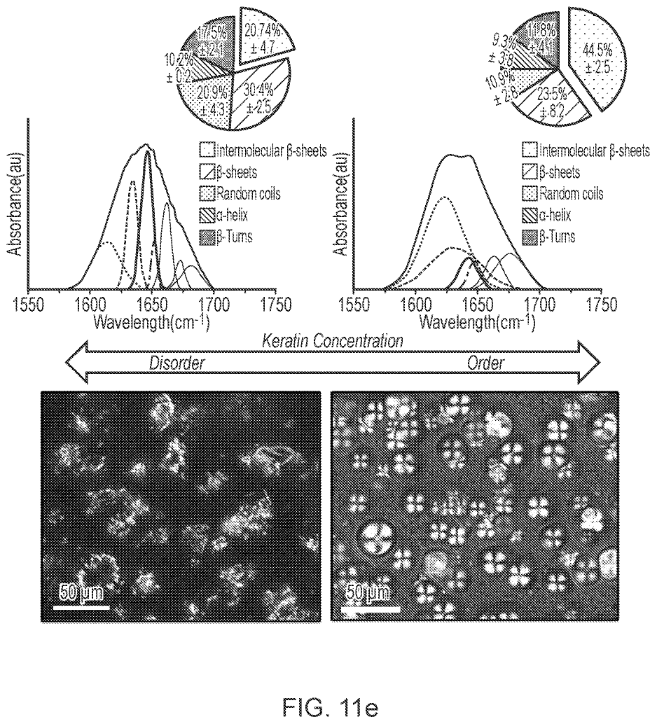

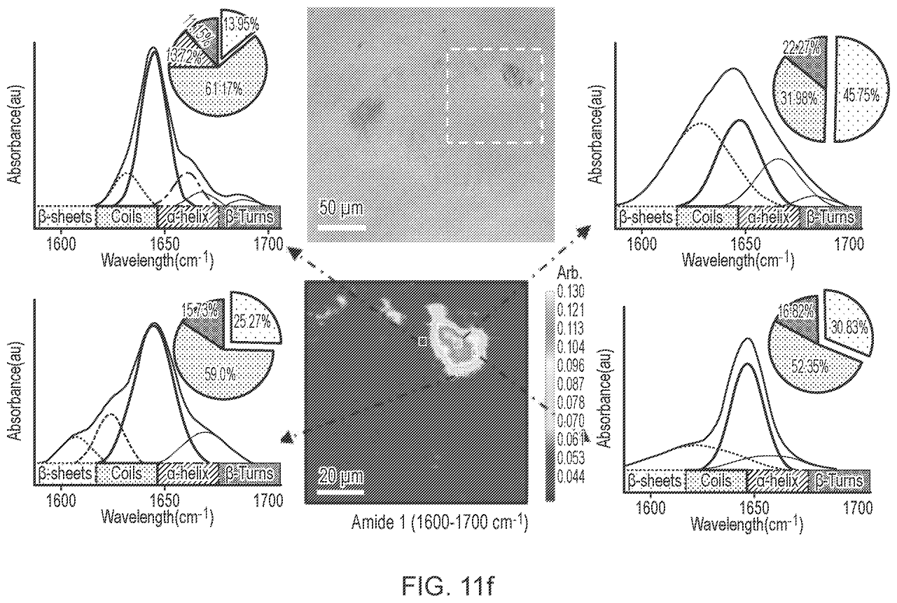

FIGS. 11a and b show organic networks of nanospheres packed on the surface of 5 wt % and 10% keratin solutions that have been allowed to dry.

These nanospheres range between 20-40 nm and have a mean diameter of 23.1 #6.64 nm, 27.97±4.75 nm, and 30.72±6.78 nm, respectively.

FIG. 10d shows the nano- and micro-fibrillar structures on the films. Different fibril populations are observed and the mean diameter of the fibrils increases with a direct proportion to the keratin concentration.

FIG. 10e shows fibrils observed on a 3 wt % keratin film crosslinked with TEGDMA. The fibrils decussate and make up from two different populations; one is ~100 nm, and the other was ~500 nm width.

FIG. 11c shows fibrils observed on a 5 wt % keratin film crosslinked with TEGDMA. The fibrils decussate and make up from two different populations; one is ~100 nm, and the other was ~500 nm width.

FIG. 11d shows fibrils observed on a 10 wt % keratin film crosslinked with TEGDMA. These fibrils are well-organized and aligned nearly parallel to each other.

FIGS. 10f and g show that a large number of spherulitic structures are seen on 10 wt % keratin film which formed large globules made of several spherulites. Some spherulites align into long strands.

FIG. 10h shows congo red staining of keratin structures in a 10 wt % keratin film. Dendritic structures were observed under white light microscopy having the same range of the organic spherulites.

To investigate keratin secondary structure conformation as a model of Intrinsically disordered proteins (IDPs) amide I spectral region deconvolution obtained from Attenuated total reflection-Fourier transform infrared (ATR-FTIR) was implemented. Secondary structures were quantified before and after inducing self-assembly, and their influence on the organization of the mineralized motifs of the different keratin films was investigated.

FTIR analysis was conducted on the keratin solutions and films using Spectrum One FTIR Spectrometer (PerkinElmer®, Buckinghamshire, UK) in ATR mode, using a white light source and an InGaAs detector. Spectra were taken between wavenumber 4000 to 700 cm−1 by averaging 32 scans per sample at a resolution of 2 cm−1. Amide I spectral region (1700-1600 cm−1) was analyzed to compare the keratin secondary structure composition using OriginPro 8.5 software (Microcal Inc.). The assignments of spectral bands were as follows: 1610-1627 cm−1 intermolecular β-sheets, 1628-1642 cm−1 β-sheets, 1643-1650 cm−1, 1643-1650 cm−1 random coils, 1650-1659 cm−1 α-helix, 1643-1650 cm−1, and 1660-1699 cm−1 β-sheets/β-turns.

The pattern and number of the organic spherulites could be modulated by increasing the keratin concentration and keeping a neutral pH (pH7).

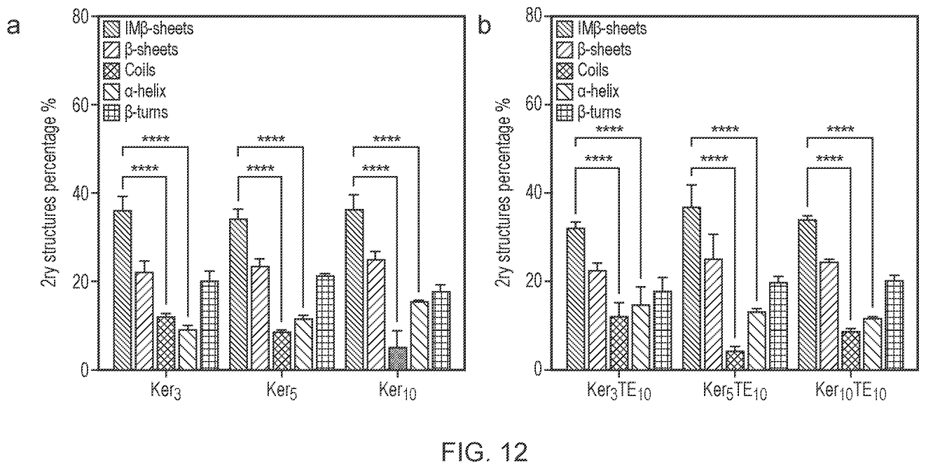

FIGS. 12a and b show that the keratin with the highest concentration, whether crosslinked or not, demonstrated an increase in the number of β-sheets over the expense of the random coils' conformation in dried films.

FIG. 11m shows that there is an increase in the number of the organic spherulites and a more definite appearance of the Maltese cross pattern in dried films with higher concentrations of keratin.

To understand the contribution of protein secondary structures in the formation of the organic keratin spherulites, Fourier transform infrared (FTIR) imaging technique was employed. Amide I band deconvolution data were analyzed of the organic spherulite and its surroundings was also carried out. Amide I band deconvolution data were analyzed of the organic spherulite and its surroundings.

FIG. 11n shows the keratin spherulites adopt mainly β-sheets, as well as random coil structures. As we move away from the spherulite, the amount of the β-sheets becomes smaller (45.75%, 30.83%, 25.27%, and 13.95%) and the random coils increase in number area (31.98%, 52.35%, 59.0%, and 61.17), α-helix was only observed in the area surrounding the spherulite (13.72%).

Thus, tuning the keratin concentration during water evaporation enabled controlled access to different keratin disorder—order ratios within the resulting films and possible control of the keratin spherulites formation.

### Example 8—Keratin Structural Tunability and Impact on Mineralization

Fourier transform infrared (FTIR) imaging was performed to investigate the changes in the secondary structure's conformation, however this time after the mineralization. To attempt this, spectra were taken from four different regions of a mineralized keratin film according to their proximity to the protein and mineralized areas. Amide I deconvolution analysis was performed.

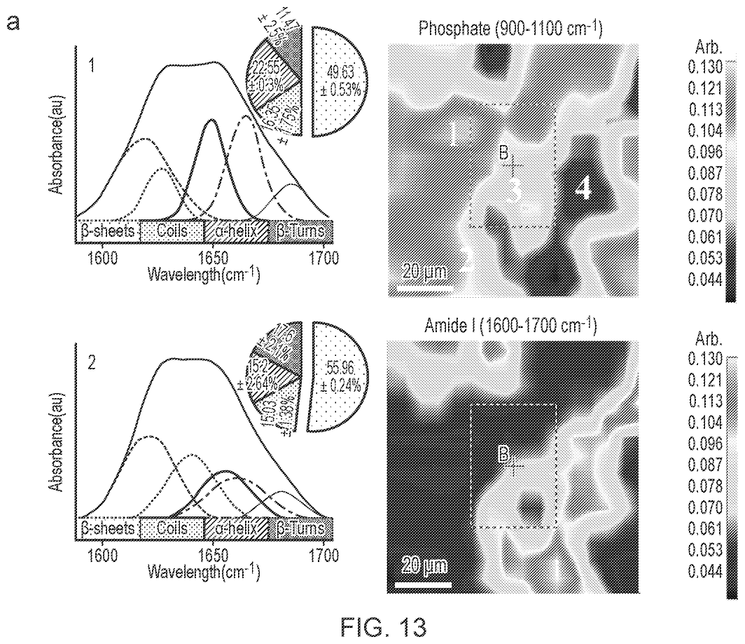

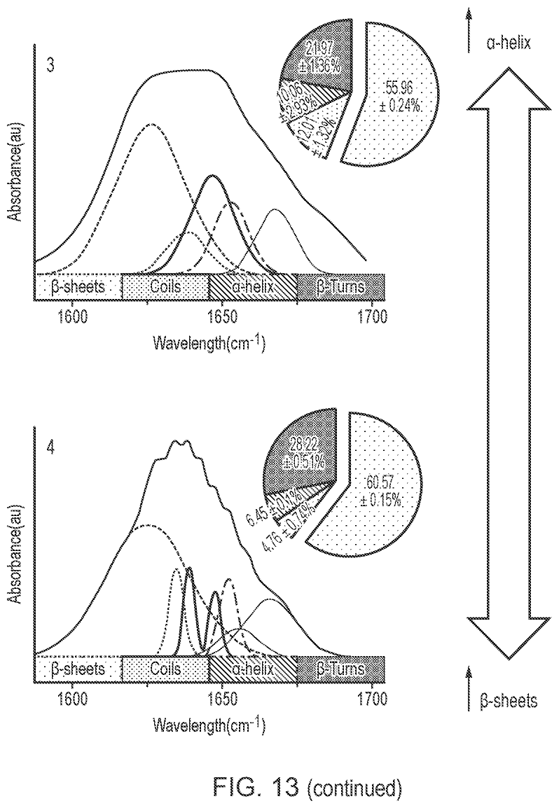

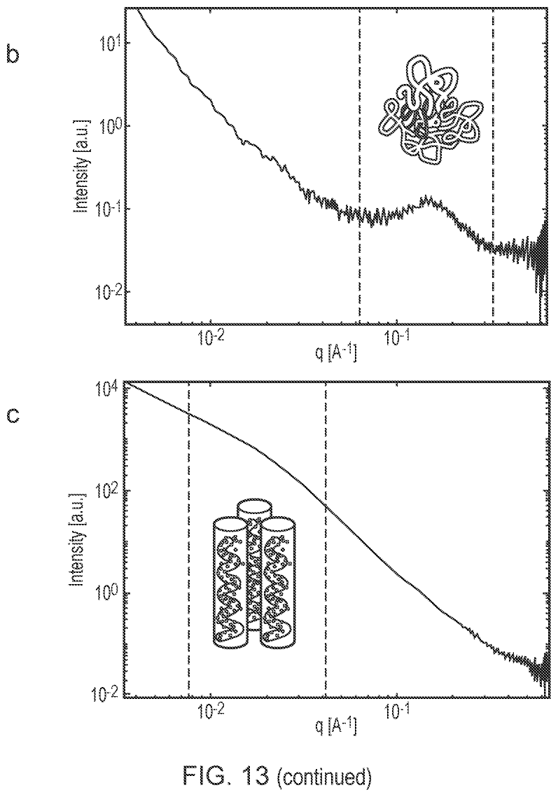

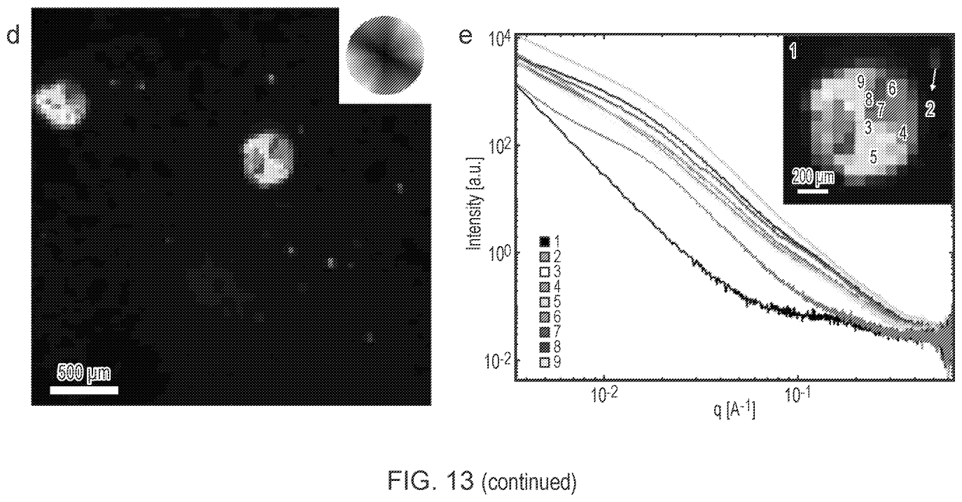

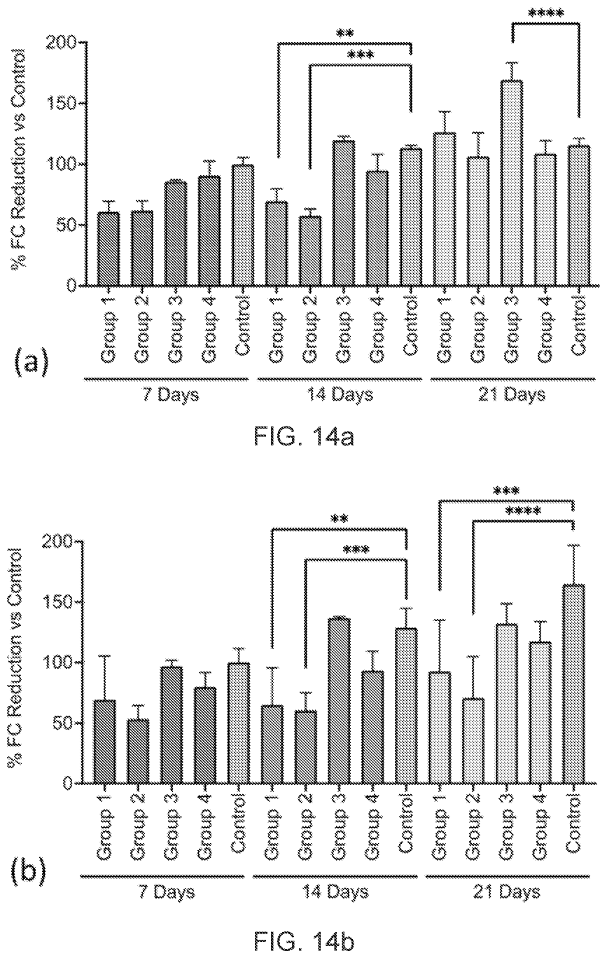

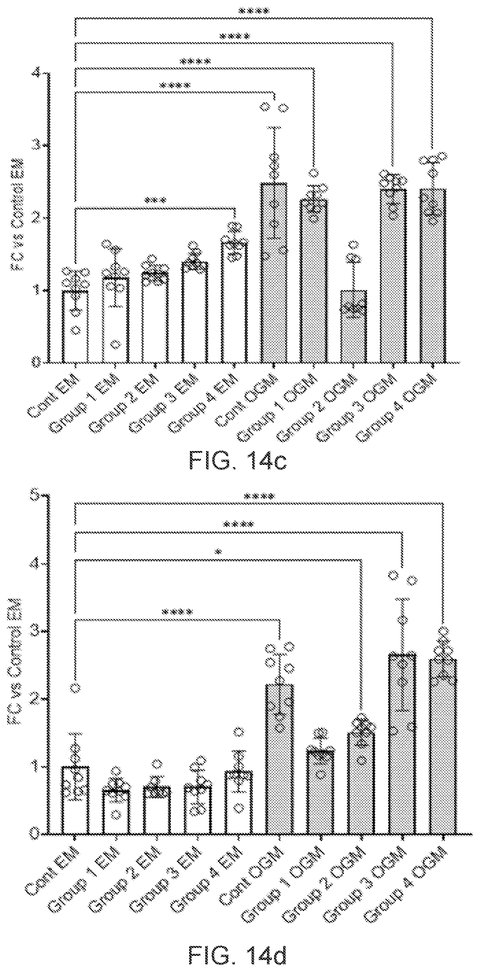

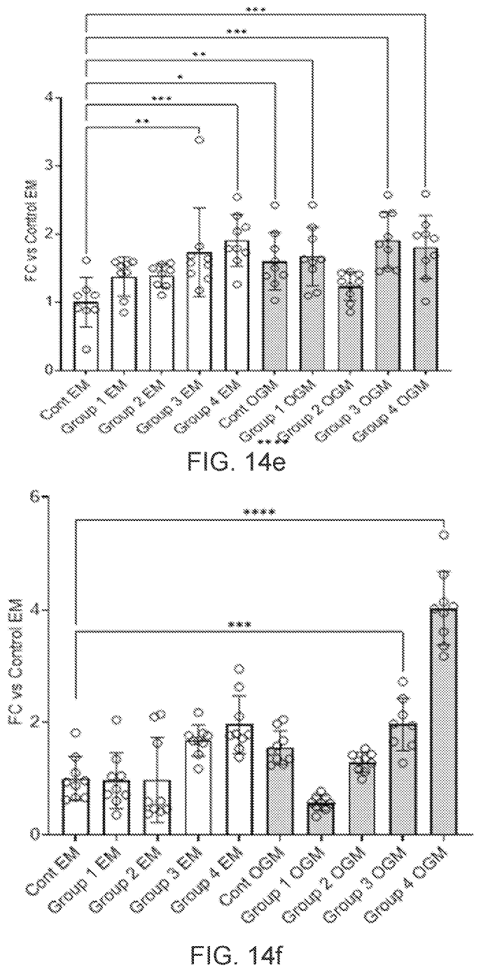

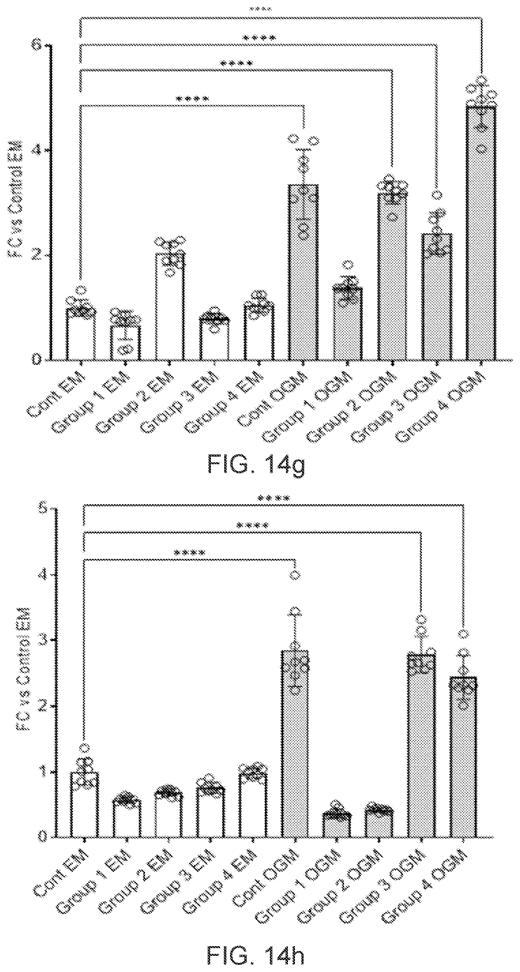

FIG. 13a shows that in close proximity to the protein, the β-sheets/turns are dominant, while as the spectra is captured away from the organic region and advances towards the mineral, there is a consistent increase in the α-helix structures and slight increase of the random coils.

These results indicate that during mineralization, the protein undergoes structural changes where β-sheets decrease and increase in a-helices and random coils could be depicted.

To evaluate the anisotropic features of the keratin films before and after mineralization, small-angle x-ray scattering (SAXS) analysis was conducted. SAXS scattering experiments were conducted at the cSAXS beamline, Swiss Light Source (SLS), located at the Paul Scherrer Institute (PSI) in Switzerland. A micro-focused X-ray beam was utilized for these experiments, with photon energies set at 12.4 keV using a Si (111) double crystal monochromator. Measurements were made using a beam size of 20 μm on non-mineralized and mineralized keratin films using a probe area of 2×2 mm. To minimize air scattering and absorption, an evacuated flight tube was positioned between the sample and the detector. The keratin film samples were mounted on a motorized stage capable of movement in two axes within the plane perpendicular to the incoming beam (x-y plane), enabling raster scanning. Fly-scanning was employed in the vertical direction (y) during the experiments, with a step size of 20 μm and an exposure time of 0.05 s. A Pilatus 2 M detector, positioned at a sample-to-detector distance of 2 m, was used to obtain the 2D SAXS patterns at each scanning point. This detector configuration covers a q-range from approximately 0.03 to 5 nm−1, where q represents the scattering vector defined as q=4nλ sin (θ), with λ denoting the X-ray wavelength and 0 representing the half scattering angle. Inside the flight tube, a 1.5 mm steel beam stop was installed to block the direct beam and protect the detector from damage.

FIG. 13b shows that the scattering curves derived from non-mineralized keratin films exhibited a prominent SAXS peak within the q range of 0.1-0.25 Å-1, indicating of a coil-like structure.

FIG. 13c shows that an additional peak within the 0.01-0.1 Å-1 range in the mineralized films, reassociated with a rod-like (helical) structure.

FIGS. 13d and e are scattering curves obtained by individual pixels from the mineralized spherulites and show a transition from a rod-like to a more coiled structure moving away from the mineral.

### Example 9—Keratin Biocompatibility Analysis Methodology

### Mesenchymal Stem Cell Expansion Media:

Low glucose Dulbecco's Modified Eagle Medium (DMEM) Sigma, 10% foetal bovine serum (FBS) (ThermoFisher Scientific, Ireland), 1% penicillin/streptomycin, 1% non-essential amino acids and 1% sodium pyruvate.

### Osteogenic Differentiation Media (OGM):

High glucose DMEM (4500 mg/L) D5671 (Sigma-Aldrich, Ireland), 10% FBS, 100 nM dexamethasone, 100 μM ascorbate-2-phosphate, 10 mM B-glycerophosphate, 1% penicillin/streptomycin, 1% non-essential amino acids and 1% sodium pyruvate.

### Membrane Pre-treatment:

Four different experimental groups and a positive control group (n=12) were used in this study: (i) 5% keratin solution (Group 1); (ii) 5% keratin solution crosslinked with triethylene glycol dimethacrylate (TEGDMA) (ratio of crosslinker to the lysine and cysteine amino-acid residues within the keratin of 1:10 (equivalent to a ratio of 100 μl keratin: 3.74 μl TEGDMA)) (Group 2); (iii) 5% keratin solution crosslinked with TEGDMA (ratio of crosslinker to the lysine and cysteine amino-acid residues within the keratin of 1:10), followed by exposure to mineralisation solution (Group 3); (iv) 5% keratin solution crosslinked with hexamethylene diisocyanate (HDI) (Group 4) and Mem-Lok® Resorbable Collagen Matrix by BioHorizons. Prior to culture, membranes were soaked in 80% (v/v) ethanol for 30 minutes at room temperature. Ethanol was removed and 3 washes with sterile phosphate-buffered saline (PBS) performed. PBS was removed and membranes were submerged in sterile DMEM until use.

Quantitative Reverse Transcriptase Polymerase Chain Reaction (qRT-PCR):

To investigate osteogenic differentiation, human bone marrow-derived stem cells (HMSCs) (P4; RoosterBio, USA) were seeded at 10,000 cells/cm² on to membranes and cultured at 37° C./5% (v/v) CO₂. Cells were allowed to attach to membranes in expansion media overnight. Subsequently, media was removed and MSCs on each membrane were cultured either in expansion media or osteogenic differentiation media. Cells were cultured for 28 days, media changed every two days, and osteogenic gene expression assessed. Briefly, media was removed, cells washed in ice cold PBS and Trizol reagent added to each membrane. Membranes were incubated in Trizol at room temperature for 10 minutes with vortexing. Chloroform was then added to samples (100 ul chloroform/1 ml Trizol) and tubes vortexed. Tubes were centrifuged at 13000 g/4° C. for 20 minutes. After centrifugation, the upper aqueous layer was removed and placed in a new microtube. An equal volume of 70% ethanol was added to these tubes and inverted 20 times. This solution was then loaded into Qiagen RNeasy columns (mini kit) and RNA extraction performed according to the manufacturer's instructions. RNA was eluted in nuclease-free water and quantified using nanodrop. cDNA was synthesised and normalised to 5 ng/ul (Quantitect Reverse Transcription Kit, Qiagen). qRT-PCR was used to assess the expression of two early osteogenic markers, runt-related transcription factor 2, (RUNX2) and alkaline phosphatase (ALP), two mid-stage markers, osterix (OSX) and bone morphogenic protein 2 (BMP-2), and two late-stage osteogenic markers, osteopontin (OPN) and osteocalcin (OCN) and was performed using Qiagen Quantitect validated primers and the Sybr Green method (Sensimix SYBR Lo-ROX mix, Meridian Bioscience) on an Eppendorf Realplex 4 Mastercycler. Gene expressions were normalised versus a housekeeping gene glyceraldehyde 3-phosphate dehydrogenase (GAPDH) and fold change gene expressions calculated versus MSCs cultured in expansion media conditions on standard tissue culture plastic.

### AlamarBlue Assay—Metabolic Viability:

At determined intervals during culture, cell culture media were removed, and cells washed with prewarmed, sterile PBS. AlamarBlue resazurin (10%, v/v; Bio-Rad) was diluted in phenol-red free media (D5030, Sigma-Aldrich) and added to each culture well. Cells were incubated in slamarBlue working solution for 4 hours at 37° C. and 5% (v/v) CO₂. After incubation, supernatant was transferred to 96-well plates and absorbances read at 570 and 600 nm to determine the metabolism of alamarBlue. The percentage of alamarBlue reduction was calculated as follows:

$$\% reduction alamarBlue = ( ( O 2 \times A 1 ) - ( O 1 \times A 2 ) / ( R 1 \times N 2 ) - ( R 2 \times N 1 ) ) \times 100$$

where O1 and O2 are the molar extinction coefficients of oxidized alamarBlue at wavelengths of 570 and 600 nm, respectively. R1 and R2 are the molar extinction coefficients of reduced alamarBlue at wavelengths of 570 and 600 nm, respectively. A1 and A2 are the observed absorbance readings for test wells at wavelengths of 570 and 600 nm, respectively. N1 and N2 are the observed absorbance readings for the negative control wells at wavelengths of 570 and 600 nm, respectively.

### Results

Mem-Lok® was used as a gold standard, positive control, as it is known to support bone regeneration in vivo. It has been shown that at least a 50% viability compared to gold standard is seen in all test membranes after culturing for seven days, regardless of culture medium used (FIGS. 7a and 7b). Improvements in viability are seen in all groups when comparing seven days to 21 days, regardless of culture medium used. Mineralisation membranes significantly outperform gold standard after 21 days in mesenchymal stem cell expansion media in terms of viability.

Favourable levels of mRNA expression of all osteogenic markers analysed (RUNX2, ALP, OSX, BMP-2, OPN and OCN) were observed with respect to all test membranes compared to gold standard.

### Example 10—Mechanical Analysis of Mineralised Keratin Membranes

### Methodology

### Enamel Sections Preparation:

Eighteen caries-free molar and premolar teeth were collected, and sections were obtained using a rotary diamond saw (XL 12205, Benetec Ltd., UK) with water cooling. The slabs were subsequently cleaned with deionised water, wiped with tissue paper, and visually examined to make sure no caries or cavities were present. The buccal surface of the samples was then placed faced down in a rectangular silicone mould and embedded into clear acrylic resin. The resin was formulated by mixing acrylic polymer beads and monomer (Oracryl™, Bracon, UK) by a 2:1 (Vol/Vol) ratio. The acrylic resin was then poured over the sections until they were completely covered, and the sections were then allowed to solidify for 30 minutes before the polishing.

### Enamel Surfaces Polishing:

Samples were polished using a polishing machine (MetaServ 3000, Buehler, USA) to obtain a flat highly polished surface for morphological analysis. Silicone waterproof abrasive paper with different grades was used. The polishing protocol was as follows: P500 for 5s, P1200 for 10s, P2500 for 10s, and P4000 for 2 minutes. The polishing direction was altered by 90 degrees and ultra-sonification being carried out after each step. Following polishing, all samples were stored in distilled water before further treatment.

### Samples Randomization:

Polished enamel sections were randomly allocated to 3 different groups (n=6): (i) 5% keratin solution (K₅); (ii) 5% keratin solution crosslinked with triethylene glycol dimethacrylate (TEGDMA) (ratio of crosslinker to the lysine and cysteine amino-acid residues within the keratin of 1:10 (equivalent to a ratio of 100 μl keratin: 3.74 μl TEGDMA)) (K₅TE₁₀); or (iii) ICON® resin infiltrant (positive control) (ICON). Each group was then divided into 3 subgroups based on the storage medium that the samples will be incubated in (deionised water (DW), artificial saliva (AS), or mineralisation solution (MS)) depending on the allocated group.

### White Spot Lesion (WSL) Induction:

Demineralisation of the polished enamel sections was performed using a bilayer acidic gel method, previously described by Zhang et al, 2018 (Zhang, J., Lynch, R. J. M., Watson, T. F. and Banerjee, A. (2018) ‘Remineralisation of enamel white spot lesions pre-treated with chitosan in the presence of salivary pellicle’, J Dent, 72, pp. 21-28). The polished enamel surfaces were then protected by an adhesive tape leaving a window 1 mm×3 mm to induce the WSLs. A demineralisation gel was prepared by dissolving 8 wt % methylcellulose powder (Sigma-Aldrich, USA) in 100° C. deionised water in a 500 ml glass beaker with continuous stirring until room temperature was maintained. Five enamel sections were then placed immersed face up inside the gel and left in 4° C. overnight to solidify. Next day, a filter paper was put over the gel and 0.1 M Lactic acid (100 ml) (Anala R, UK) pH 4.6 was added on top followed by incubation at 37° C. for 7 days. After 7 days, samples were taken out and WSLs were then rinsed with deionised water for 1 minute to remove any remaining gel.

### Storage Media Preparation:

No additions were made to the deionised water (DW) before use for the DW storage media.

An established protocol was used to formulate and prepare 1 litre of artificial saliva for experimental use. 0.1029g of calcium chloride dihydrate, 0.01904g of magnesium chloride, 0.544g of potassium dihydrogen phosphate, 5.206g of 4-(2-hydroxyethyl)-1-piperazineethanesulfonic acid, and 2.2365g potassium chloride were added and mixed thoroughly with an orbital shaker to 1 litre of deionised water. The solution was titrated to pH 7.0 (±0.05) using 0.1M of sodium hydroxide and measured using a pH meter (Mettler Toledo). The solution was covered using (Bemis Parafilm M) and stored at room temperature.

The following protocol was used to make mineralisation solution: 600 ml of mineralisation solution was prepared using 0.60276 g of hydroxyapatite powder, 0.0084 g of sodium fluoride, and 1.2554 g of bis-tris methane by the following steps. In fumigated conditions, bis-tris methane was dissolved in 35 mL of deionised water and adjusted to pH 6.0 (±0.05). Sodium fluoride and hydroxyapatite powder were subsequently added to 500 ml of deionised water. 69% nitric acid was added dropwise with a micropipette to allow the hydroxyapatite and sodium fluoride powers to fully dissolve at a pH 2.4. The solution was titrated using the bis-tris buffer to achieve a pH of 6.0 (±0.05). The solution was covered using parafilm (Bemis Parafilm M) and stored at room temperature.

### Applying Treatments to WSLs:

Keratin was extracted from sheep wool as previously mentioned in Example 1. K₅ samples were prepared by adding 5 wt % keratin to deionised water. K₅TE₁₀ samples were prepared by adding 5 wt % keratin to deionised water and then TEGDMA was added as a crosslinker to bind to the lysines and cysteines of the keratin. All mixtures were then drop casted on the WSLs surfaces according to the previously allocated groups and were then allowed to dry at 37° C. for 5 minutes before placing them into the allocated storage media.

The white spot lesion sections that received ICON resin infiltration were treated with 99% pure ethanol (ICON dry DMG) which was left for 30 seconds on the white spot lesion surface. Following this the surface was cleaned with oil free compressed air. The lesion surface was then treated with a TEGDMA-based resin matrix (ICON®—infiltrant from DMG) which was left on the surface for 3 minutes. To maintain a wet surface, additional infiltrant was applied to the surface as required. The infiltrant was then dispersed with oil free compressed air and then light cured for 40 seconds with UV light at 200-400 nm wavelength. Additional infiltrant was then applied onto the surface for 1 minute and again dispersed with oil free compressed air before being light cured for a further 40 seconds.

### Microhardness Measurements:

Surface microhardness was carried out on all samples by a microhardness tester fitted with a Knoop diamond indenter (Duramin, Struer, Denmark). For each sample, 5 indentations were measured with a spacing of at least 100 μm. A load of 0.2 kilogram-force (kgf) and dwell time of 10 seconds were used to measure the untreated polished enamel and untreated WSLs. For the intervention groups, after the indentation had been prompted, samples were imaged on white light microscope (Keyence, America) and microhardness measurements were then calculated using the formula HK=14.299×F/D², where F is the applied force in newtons (N) and D is the length of the indentation (mm).

### Scanning Electron Microscopy (SEM):

After microhardness measurements had been conducted, cross-sections of the different groups were obtained and were then etched with phosphoric acid 35% for 20 seconds and rinsed for 20 seconds for removing the smear layer and preparing them for SEM imaging. Samples were mounted on aluminium stubs with double-sided carbon tape and were then sputtered with 8 nm conductive gold coating (Leica EM ACE600 sputter coater, Milton Keynes, UK). SEM images were obtained using a Schottky field-emission JSM-7610F SEM (JEOL Ltd., Hertfordshire UK), with the surface topography being observed using the secondary electron detector to assess the changes in morphology of the samples before and after treatment.

### Statistical Analysis:

The results were statistically analysed using Graphpad Prism software. Shapiro Wilk test showed that the data was normal, and one way Analysis of variance (ANOVA) was used to compare microhardness of all groups. Tukey's HSD post-hoc test was used to assess the significance between the groups at p<0.001.

### Results

### Microhardness Measurements:

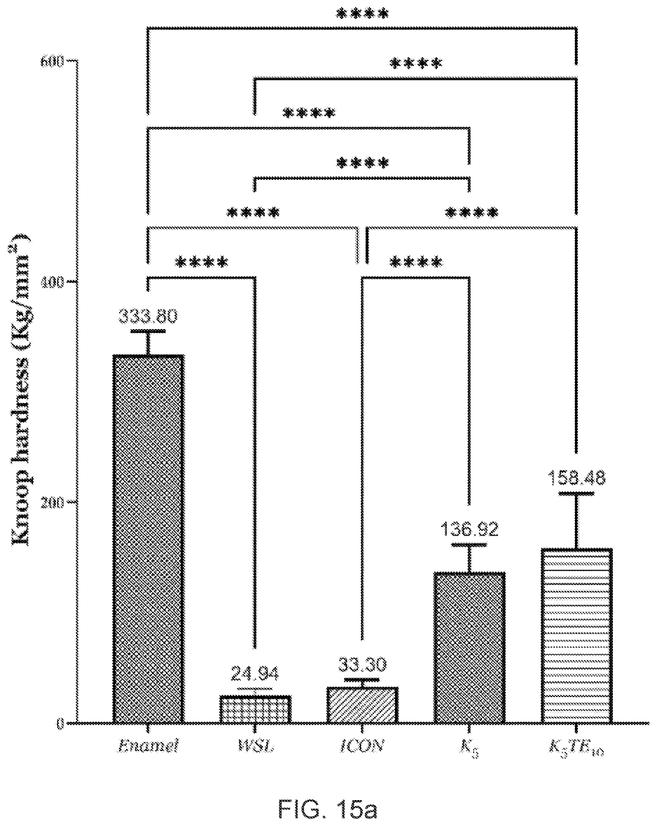

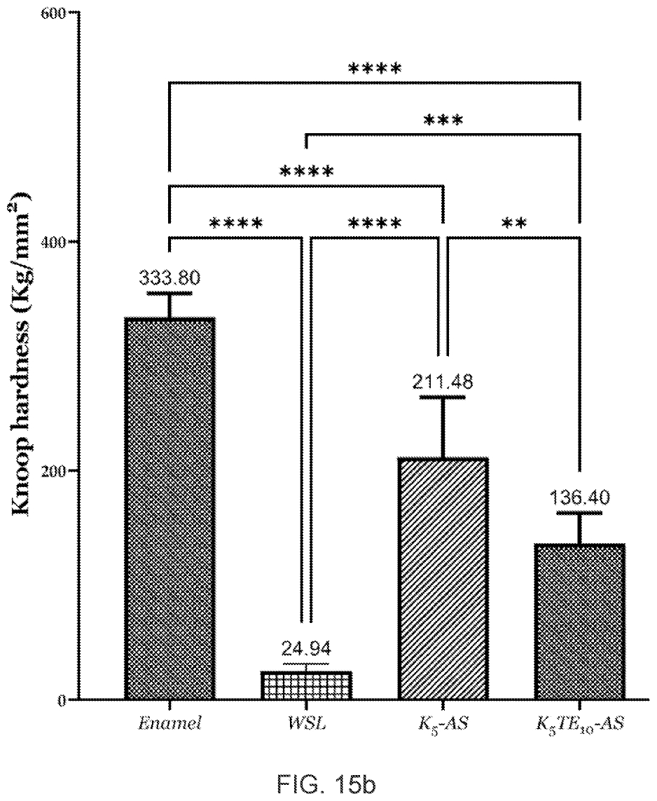

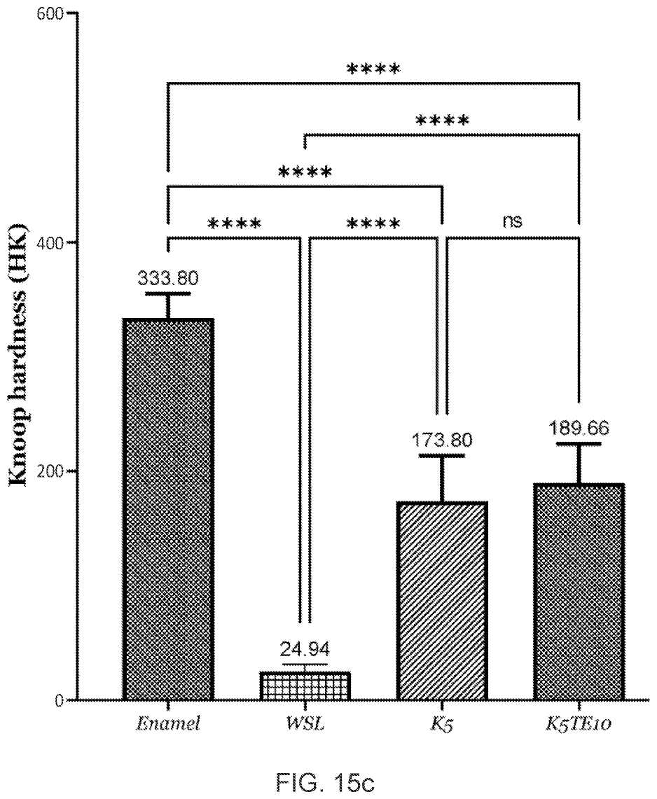

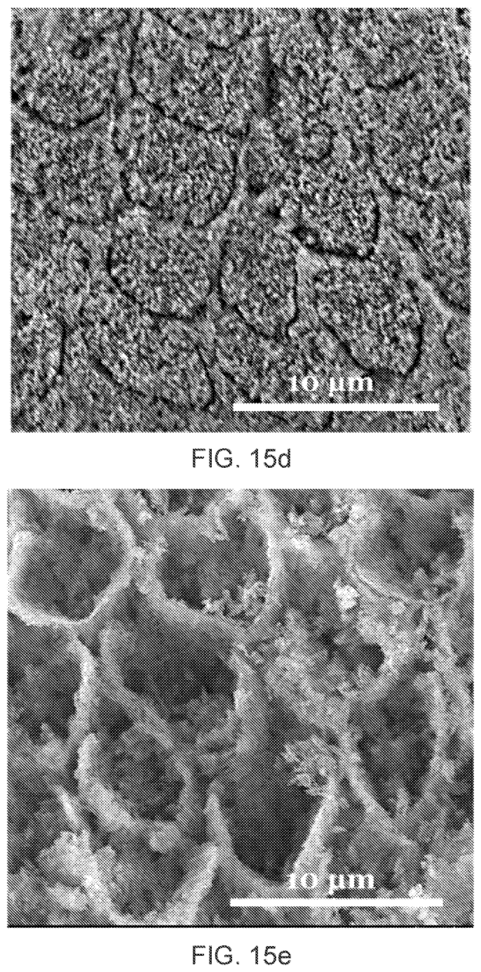

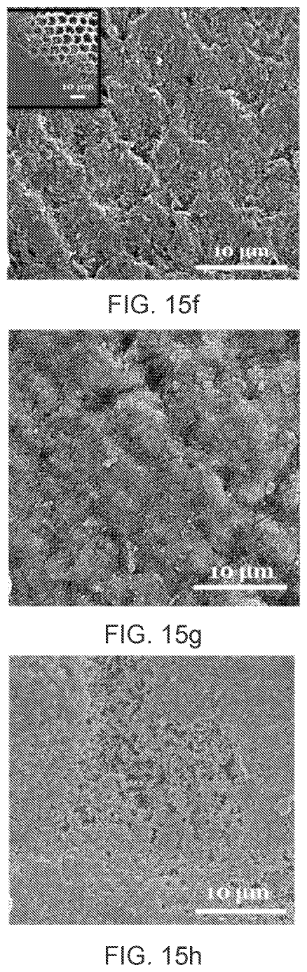

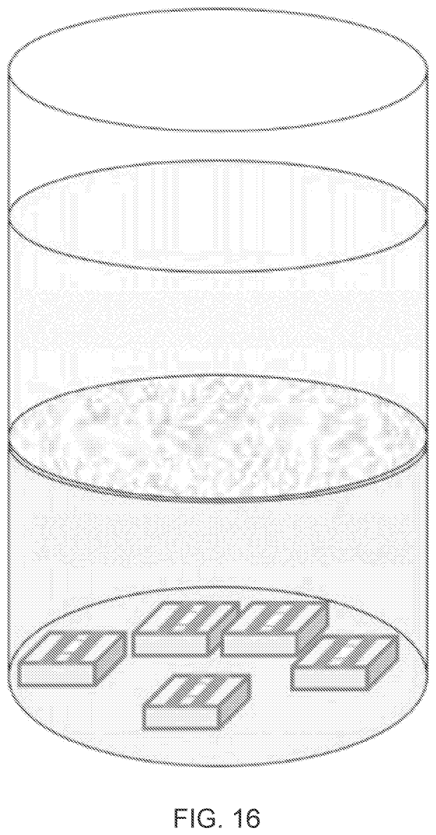

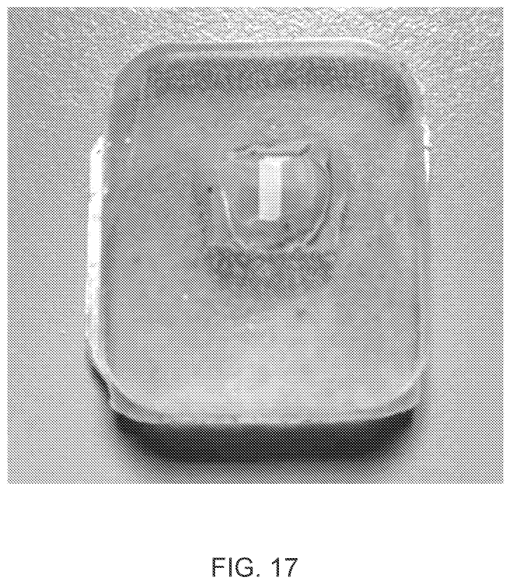

As shown in FIG. 15a with respect to DW storage medium, polished enamel and WSLs means were 333.80±24.65 kg·mm⁻² and 24.94±6.16 kg·mm⁻², respectively. WSLs treated with ICON showed Knoop microhardness of 33.3±21.09 kg·mm⁻², however, K₅ and K5TE10 showed mean microhardness of 136.9±24.65 kg·mm⁻² and 158.48±45 kg·mm⁻².

As shown in FIG. 15b with respect to AS storage medium, polished enamel and WSLs means were 340.8±19.7 kg·mm⁻² and 27.36±6.05 kg·mm⁻², respectively. WSLs treated with ICON showed Knoop microhardness of 44.68±11 kg·mm⁻², however, K5 and K5TE10 showed mean microhardness of 222.48±48.5 kg·mm⁻² and 136.4±26.62 kg·mm-2.

As shown in FIG. 15c with respect to MS storage medium, polished enamel and WSLs means were 350.4±53 kg·mm⁻² and 11.2±3.3 kg·mm⁻², respectively. WSLs treated with ICON showed Knoop microhardness of 13.2±1.17 kg·mm⁻², however K5 and K5TE10 showed mean microhardness of 173.8±39.6 kg·mm⁻² and 144.24±50.419 kg·mm-2.

To summarize, polished enamel surfaces showed significant difference compared to all other samples. In all storage media, ICON treated WSLs showed no significance with the WSLs, nevertheless, significant difference was observed between both keratin treated lesions compared to WSLs and ICON with keratin groups exhibiting improved hardness.

### SEM Analysis:

An SEM image of the polished untreated enamel is shown in FIG. 15d and an SEM image of the WSLs is shown in FIG. 15e. The microstructures of intact enamel consists of prism and inter-prism continua and tightly packed hydroxyapatite crystals. However, in the WSLs the prism cores have been significantly affected and there is a loss in the hydroxyapatite crystallites and their organization.

K₅ treated WSLs incubated for 7 days in different media were observed under SEM (FIGS. 8f, 8g and 8h). In DW, keratin infiltrates the WSLs and forms a coat covering the lesion. Keratin incubated in AS forms a homogenous newly-grown mineral layer after remineralization where nanorods are oriented parallel to each other. Keratin treated WSL incubated in MS also has a homogenous repaired mineralized layer of enamel-like organized apatite crystals around 50-70 nm in width, some of which are tightly packed bundles with specific orientation.

### Example 11—White Spot Lesion Study

### Aim

To study the potential of enamel-like crystal remineralisation in infiltrated carious white spot lesions with a novel clinically friendly keratin-based membrane.

### Methodology

Enamel sections (n=18) from caries-free molar and premolar teeth were embedded in acrylic resin, polished, randomized into 3 groups and were then immersed in a demineralization solution for 7 days to induce white spot lesion (WSL) (FIGS. 9 and 10). All samples were then etched with 15% HCl for two minutes to allow better infiltration of the treatments and rinsed with deionised water for 30 seconds, then dried with oil-free air using an air compressor. Six samples were assigned for each group, the groups were assigned as follows: positive control (ICON® resin infiltrant), and two different test groups. ICON® is a WSL treatment provided by DMG Chemisch-Pharmazeutische Fabrik GmbH comprising camphoro quinone as a photoinitiator and tri-ethylene glycol dimethacrylate (TEGDMA). In the first group, samples were treated by pipetting 50 μl of 5 wt % keratin dissolved in Milli-Q water on WSLs. Whereas the other test group samples were treated by pipetting 5 wt % keratin dissolved in Milli-Q water and crosslinked with TEGDMA (ratio of crosslinker to the lysine and cysteine amino-acid residues within the keratin of 1:10 (equivalent to a ratio of 100 μl keratin: 3.74 μl TEGDMA)). All Samples were then incubated at 37° C. for 2 minutes to allow the keratin solution to form hydrogels inside the lesions before immersion into the allocated storage medium, either deionized water (DW) or artificial saliva (AS) at 37° C.

Visual assessment using the International Caries Detection and Assessment System (ICDAS) criteria was used for analysis of the WSLs together with measuring the grayscale value changes pre- and post-intervention using white light microscopy (WLM), and optical coherence tomography.

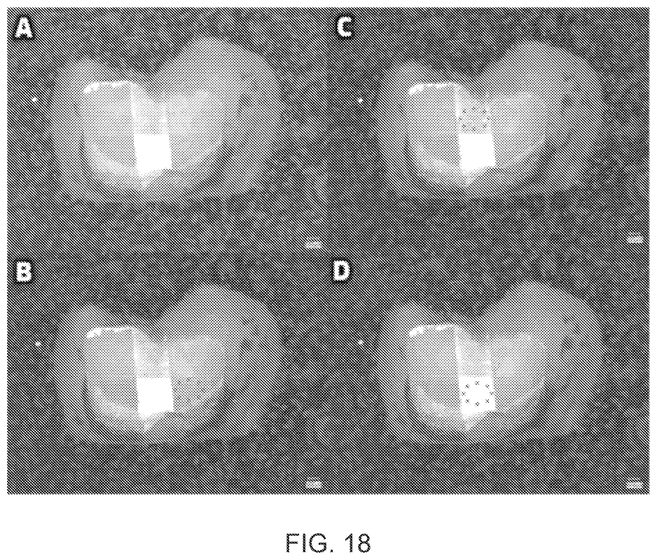

WLM was used to obtain surface characterization before and after each treatment and in cross-section and to aid the outcome of the ICDAS scoring method. Images were obtained using an E20 lens at x20 magnification. ImageJ (NIH, US) was used for grayscale analysis of the data. All images were compared, and it was decided a 400×300 pixel oval selection area was sufficient for grayscale data capture as it captured enough pixel information without being influenced by the borders or transition surfaces of the lesion. The selection area was dragged over each area of interest and grayscale values were recorded for each pixel (FIG. 18).

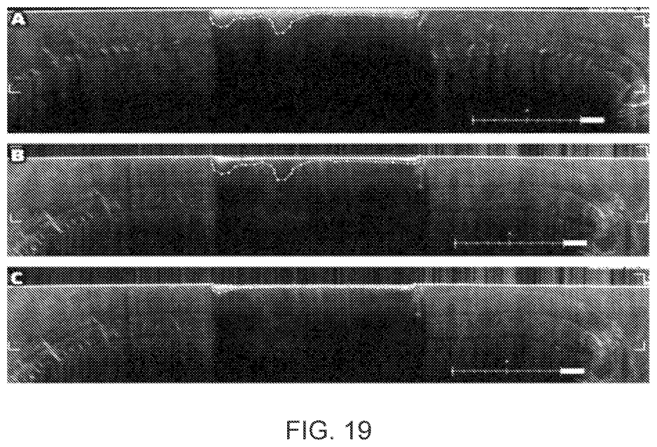

Optical coherence tomography (OCT) (VivoSight, Kent, UK) was also used to measure samples at baseline, and WSL lesion depth and surface area density before and after treatments. The scan area was 6 mm wide to include the entire lesion cross-section. Multi-slice mode was used with a 0.001 mm interval between each slice. 2-Dimensional cross-sectional images of the control and intervention areas were captured for each sample. Images were then subsequently transferred to ImageJ (NIH, US) for grayscale analysis. The imaging analysis procedure is illustrated in FIG. 19.

### Results

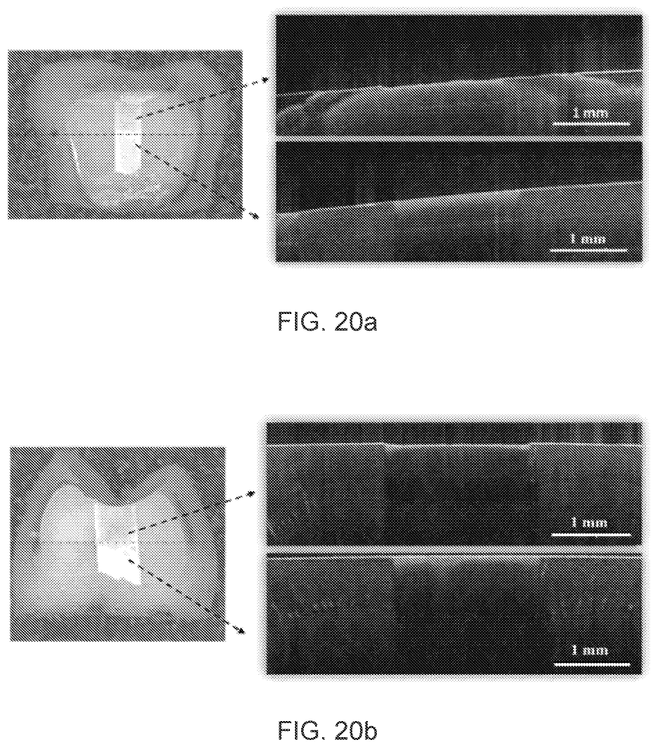

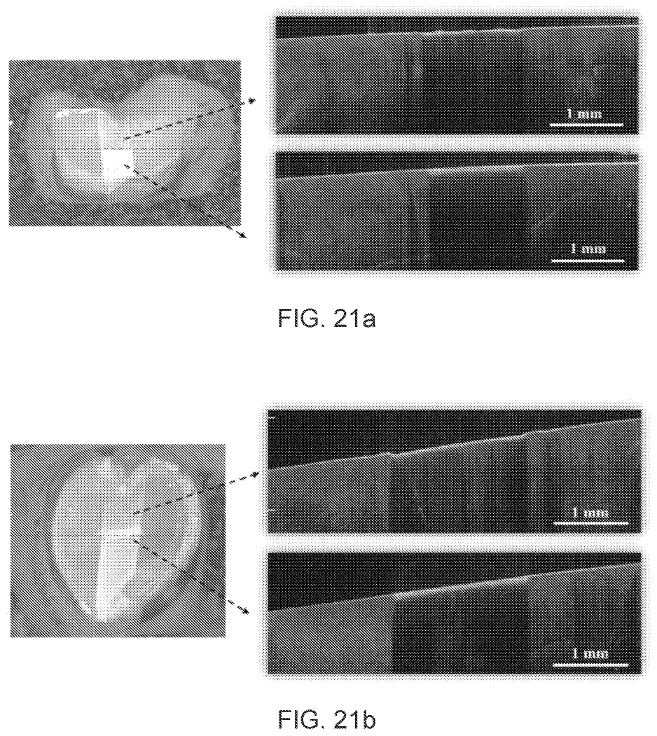

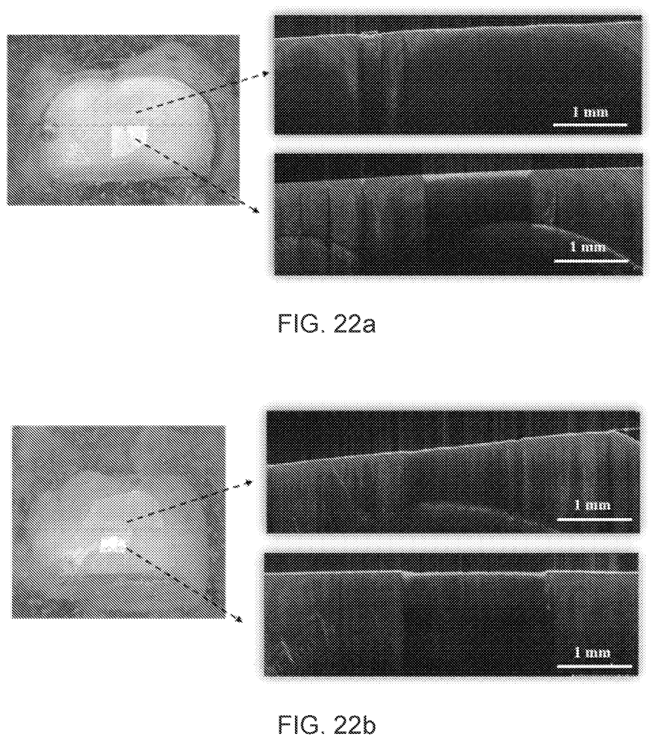

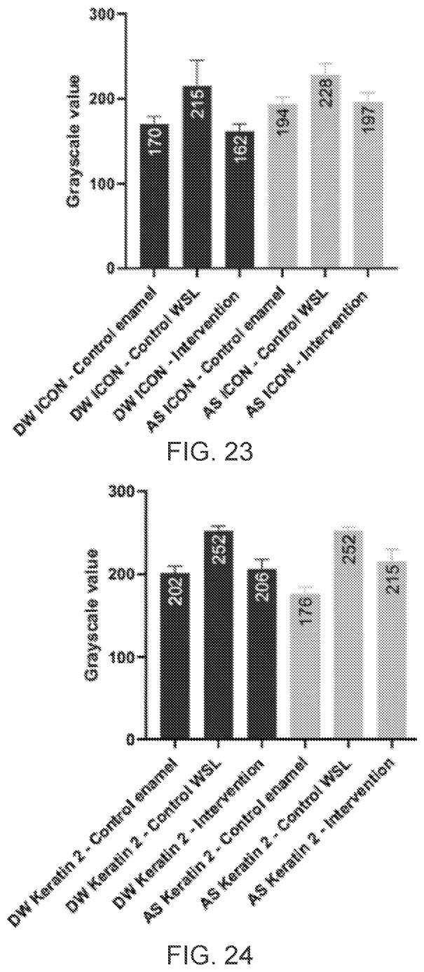

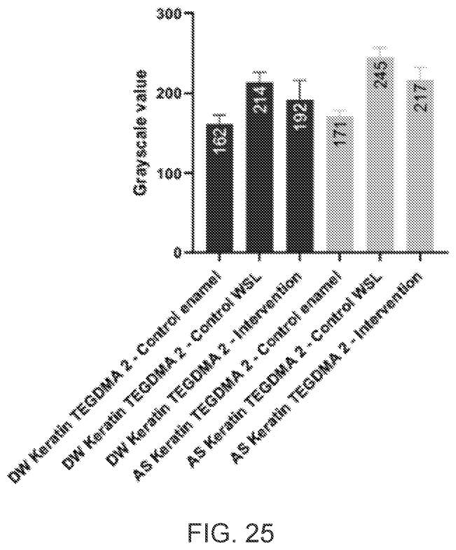

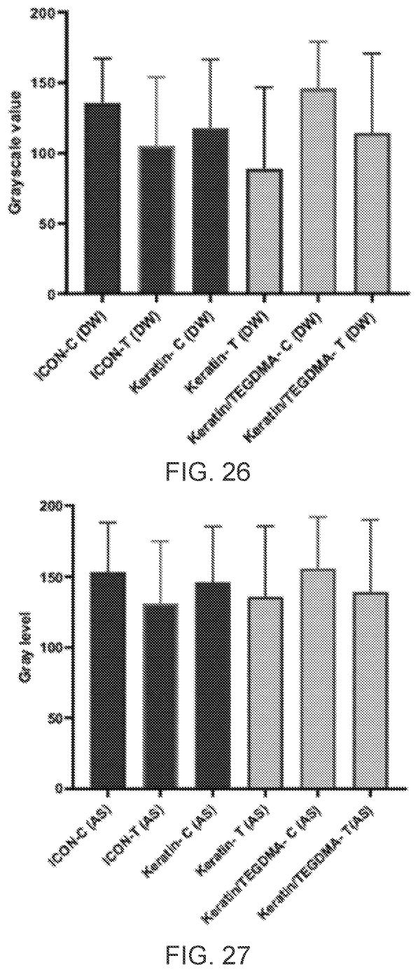

OCT and WLM grayscale value changes were plotted following image analysis as shown in FIGS. 20 to 22. OCT and Keyence analysis highlighted consistent grayscale value differences between the control and intervention areas of each sample (FIGS. 23 to 27). Both wool-derived keratin test groups showed lesion depth and density reductions, also reduction in grayscale values were detected after image J analysis when both keratin solutions were applied comparable to the positive control (ICON®) in either deionized water or artificial saliva.

The results show that membranes are effective in treating white spot lesions to a similar extent as the gold standard, ICON® resin infiltrant. Furthermore, it is believed that as the keratin membranes promote remineralisation on the teeth (unlike current treatments such as ICON®) and in doing so would provide additional long-term benefits as well as short term benefits.

### Example 12—Induced Enamel Lesion Surface Characterization

An in-vitro pre-clinical trial was performed using induced enamel WSL models to investigate the potential of keratins to treat the artificially induced enamel pores and restore their mechanical properties.

Extracted human non-carious molar teeth (with HRA approval from NHS Research Ethics Committee, reference number: 16/SW/0220) were examined under white light microscope (GXM-XPLPOLTEC-5, UK) to make sure no caries or cavities were present. The facial surface of the samples was then placed faced down in a silicone mould and were then embedded into clear acrylic resin (Oracryl™, Bracon, UK). Enamel sections were obtained using a water-cooled rotary diamond saw (XL 12205, Benetec Ltd., UK). Sections were carefully polished using a polishing machine (MetaServ 3000, Buehler, USA) by the aid of silicon carbide grinding papers (Struers®, UK) from coarse to fine as follows (P500, P1200, P2500, and P4000). The polishing direction was altered by 90 degrees and ultra-sonification was carried out after each step. Following polishing, all samples were stored in distilled water before further treatment.

Polished enamel surfaces were covered with tape to leave a window approximately 1 mm wide and 2 mm long on the facial surfaces of each molar. WSLs were then induced on the window using an established protocol. For comparison, we symmetrically divided all WSL windows into two parts; the bottom was protected by an adhesive tape to act as a negative control, and the top was used for applying the repair treatment and was pre-etched with 15% HCL for two minutes to open up the enamel pores and ensure the infiltration of treatment and were washed with deionized water, sonicated for 2 minutes in water bath to remove any residual contaminants, air dried, and stored at 4° C. until used.

WSL blocks were treated with either Kers or resin infiltrant (ICON®, DMG America). Each treatment group was incubated in three storage media; UPW, artificial saliva, or mineralization solution. For the keratin treatments, similar fabrication procedures were undertaken as mentioned previously and were pipetted (20 μl) on the uncovered area to infiltrate the white spot lesions then left to dry and subsequently stored at 37° C. for 7 days. Enamel treated blocks were later washed with UPW, sonicated for 2 minutes to remove debris, and air dried.

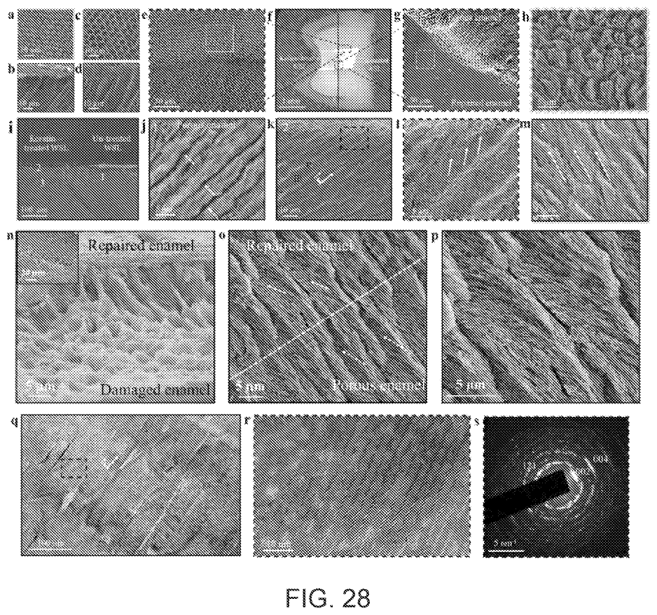

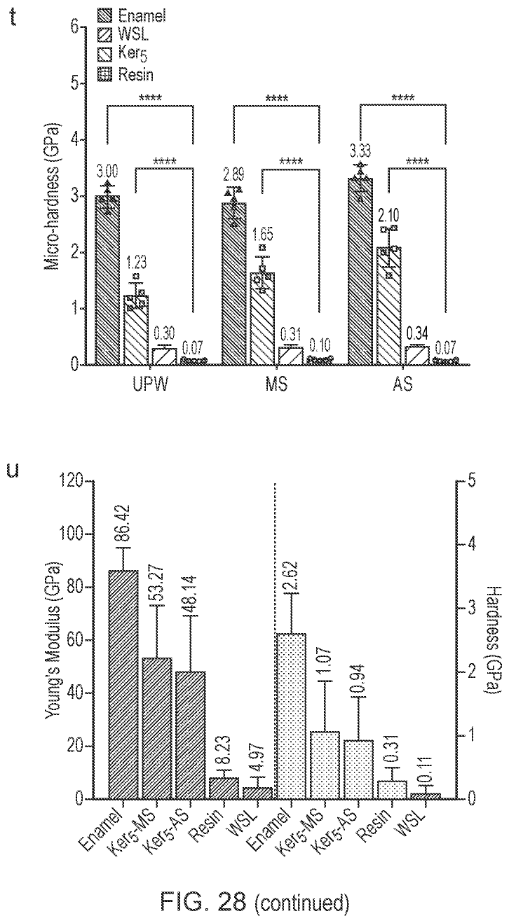

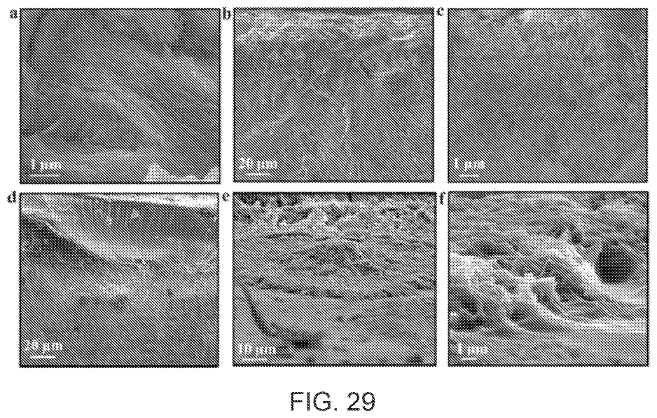

FIGS. 28a to 28e show scanning electron microscopy images that validate WSL induction.

FIGS. 28a and 28b show the microstructure of intact polished enamel constitutes the prism, inter-prism continua, and the tightly packed hydroxyapatite crystals

FIGS. 28c and 28d show the prism cores have been significantly affected and a loss of the hydroxyapatite crystallites creating gaps between prisms and loss of the crystal organization.

FIGS. 28f to 28i show improvements in the lesion color and appearance after treatment of the WSLs with Keratin.

FIGS. 28g to 28m show the films incubated in mineralization solution from the surface of the treated lesion and demonstrate signs of enamel reconstruction. The gaps and porosities that were created through both the prisms and inter-prisms as a result of inducing the WSL in the enamel (FIG. 28j) are filled with new nanocrystals along almost the whole depth of the treated WSL (FIG. 28k-m).

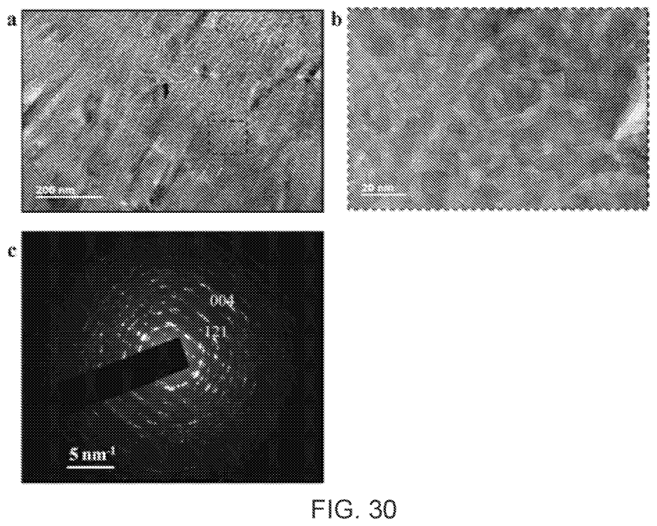

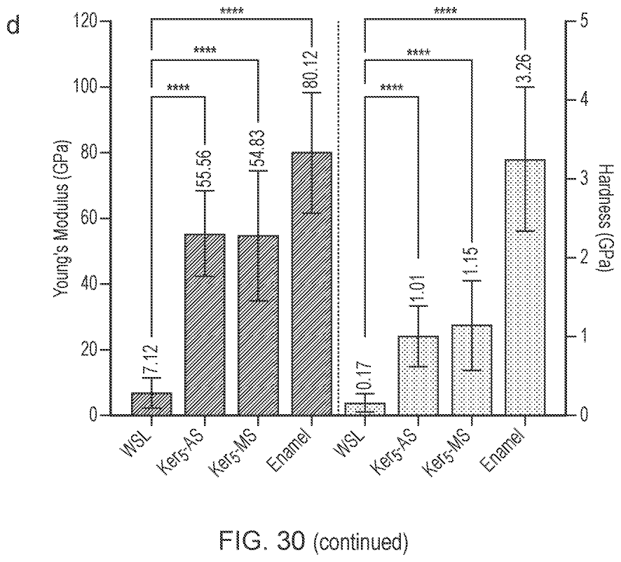

FIG. 30a shows integration between the HA crystals and the keratin at the surface where the keratin seems to infiltrate between the mineral crystals and guide their growth.

FIGS. 28n to 28p shows that similar results are observed when the teeth were incubated in artificial saliva, where lesion gaps were repaired with enamel-like nanocrystals that developed a continuation from the lesion crystals and deeply filled the pores.

FIG. 30b shows that when keratin treated WSL were incubated in UPW, they formed only a coating on the surface with no evidence of mineral repair.

WSLs were also investigated without adding any treatment after remineralization in mineralization solution.

FIG. 30c shows that when no treatment is added after remineralization in mineralization solution, mineral precipitates are deposited over the surface without a definite orientation or following the enamel prism orientation.

FIG. 30d is an SEM image showing resin infiltrant incubated in mineralization solution filling the gaps of the porous enamel and formed a layer of the resin on top of the WSL.

FIGS. 30e and 30f show that the mineralization detected within the resin-infiltrated enamel prisms exhibited clumps on top of the resin with no defined crystal features.

Furthermore, focused ion beam (FIB) lamella of the keratin treated lesion was milled to investigate the crystalline phase. High-resolution transmission electron microscopy (HR-TEM) of the keratin treated lesions were captured from an area near to the top of the enamel surface and another one at the bulk ~50 μm away from the surface.

FIGS. 28q to 28s and FIGS. 30a to 30c are TEM images that reveal bundles of nanocrystals, where the newly formed nanocrystals were detected between the native crystals oriented parallel to their growth direction along the c axis at both locations and integrated between the prisms showing a continuous pattern and selected area electron diffraction (SAED) diffraction also confirmed the apatite phase.

These results demonstrate the capability of the keratin to repair the defective enamel within almost the whole depth of the lesion.

To assess the mechanical properties of the reconstructed enamel, microhardness analysis was carried out.

Microhardness analysis was performed as follows. Surface microhardness was carried out on all samples by a microhardness tester fitted with a Knoop diamond indenter using the microhardness tester (Duramin-20, Struers Ltd, Rotherham, UK). Microhardness measurements of sound enamel were obtained from the polished enamel around the WSLs window within the experimental groups. For each sample, 5 indentations were measured with a spacing of at least 100 μm. A load of 0.2 kgf and dwell time of 10 s were used to measure the untreated polished enamel and untreated WSLs. For the intervention groups, after the indentation had been prompted, samples were imaged on white light microscope white light microscopy (VHX-7000 series, Keyence, America) and microhardness measurements were then calculated using the formula below. HK=14.299×F/D2; F=the applied force (N); D=the large diagonal (mm).

FIG. 28t shows the Knoop hardness measurements of the healthy enamel and the untreated WSLs were first recorded in the different storage media. In ultra-pure water (UPW), keratin treated lesions showed microhardness of 1.23±0.22 GPa, compared to 3±0.19 GPa in enamel, 0.07±0.02 GPa in WSLs, and 0.3±0.05 GPa resin infiltrant. In mineralization solution, Keratin 5 wt % microhardness was enhanced to 1.65=0.3 GPa after mineralization, while enamel and WSL hardness were estimated to be 2.89±0.28 GPa and 0.1±0.1 GPa. Also, Keratin 5 wt % showed significant improvement of the microhardness in artificial saliva of 2.1±0.35 from 0.1±0.02 GPa in WSLs. On the other side, resin infiltrant has not shown a noticeable improvement, where the hardness was estimated to be 0.34±0.03 GPa in lesions incubated in mineralization solution and 0.31±0.05 GPa in lesions incubated in artificial saliva.

Young's modulus and hardness measurements were also taken. Keratin films and enamel samples were glued onto an aluminum holder. Nanoindentation tests were carried out by nanoindenter (iNano) by Nanomechanics, Inc. (maximum indentation load of 50 mN). The mechanical properties of the mineralized films were recorded with an indentation depth of 30 nm on the films in order to decrease the organic tissue influence underneath. Enamel samples Young's modulus and hardness were evaluated following a previously established method45 with a maximum depth of 1000 nm with 50-100 indents were made for each sample.

FIG. 28u shows improvement in the stiffness and elastic modulus of enamel before and after repair by keratin in artificial saliva and mineralization solution were also evaluated with nanoindentation measurements. The mean stiffness and hardness of enamel before inducing the WSL were estimated to be 86.42±8.67 GPa, and 2.62±0.67 GPa, respectively. While after WSL induction, lesions demonstrated 4.97±3.45 GPa in Young's modulus, and Hardness was evaluated to be 0.11±0.12 GPa. After keratin treatment was introduced inside the defective enamel, the modulus and hardness has shown significant improvements of 53.27±19.78 GPa and 1.07±0.78 GPa, respectively. Similar results were observed in lesions treated with keratin in artificial saliva, where repaired enamel demonstrated enhanced stiffness of 48.14±21.16 GPa and 0.94±0.68 GPa hardness. Resin treated lesions demonstrated a modulus of 8.23±2.95 GPa and hardness of 0.31±0.2 GPa, which is in agreement with previous studies.

FIG. 30d shows nanoindentation measurements recorded from the bulk of the sample confirmed the improvement in the mechanical properties.

The previous results complement the SEM and the HR-TEM findings that the keratin has the potential to treat and infiltrate almost the whole depth of the induced enamel lesions.

### Example 13—Animal Study to Assess Keratin Potential for Bone Healing

The inventors went on to investigate the potential of the keratin membranes to induce bone healing in an in vivo study.

A total of 42 healthy male rats (~250-350 grams), aged 6-8 weeks were used. Surgery was performed to create a calvarial defect in each rat. To create the calvarial defect, a 2 cm longitudinal incision was created along the midline of the skull. The periosteum will be carefully incised, elevated, and retracted bilaterally for full exposure of the calvaria. A 6.0 mm diameter trephine burr was used to drill a round segmental calvarial defect in the parietal bone of each rat with caution not to damage the dura during removing the full thickness of the defect. During the drilling, the area was continuously irrigated with sterile saline irrigation and subsequently the calvarial disk was carefully removed using tweezers.

The defects were then randomly allocated to the 6 different groups. Each rat provided one calvarial defect (7 defects per group).

4 different experimental groups of Keratin membranes were tested:
5 wt % keratin Keratin membrane (Ker5),
Keratin crosslinked with Triethylene Glycol Dimethacrylate (Ker5TE1)
Mineralized keratin-TEGDMA (M-Ker5TE1)
Keratin-HDI (Ker5H3).

A positive control (collagen membrane) and a negative control (empty defect), to assess natural healing without treatment, were also included.

Membranes were sterilized for 20 min under ultraviolet inside a sterilized hood before the surgical intervention. Hydrated membranes were implanted covering the defect with the extreme of the membranes fixed under the periosteum. The skin was then closed over in layers with 4-0 Vicryl sutures. Animals were monitored daily until euthanasia for any complications or abnormal behaviour.

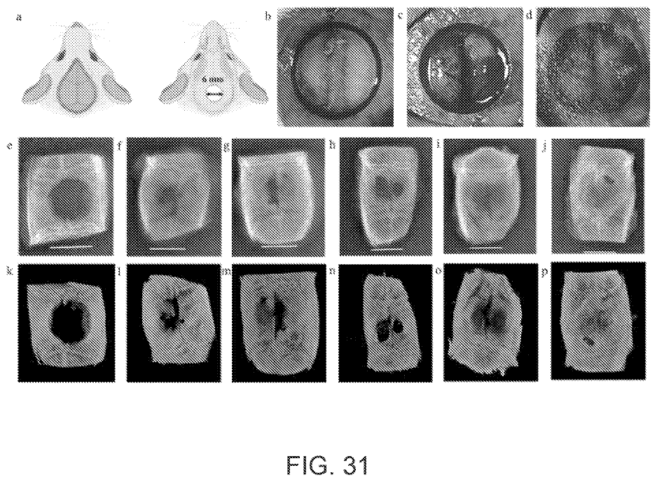

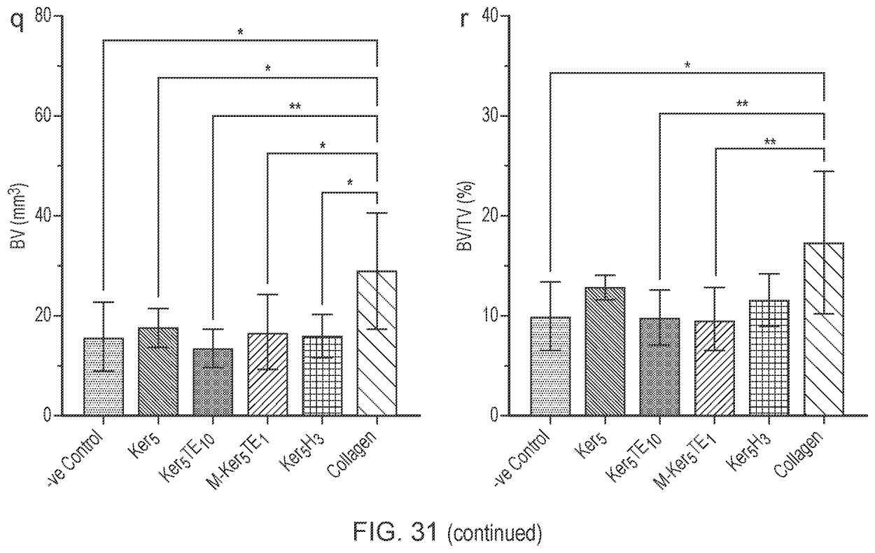

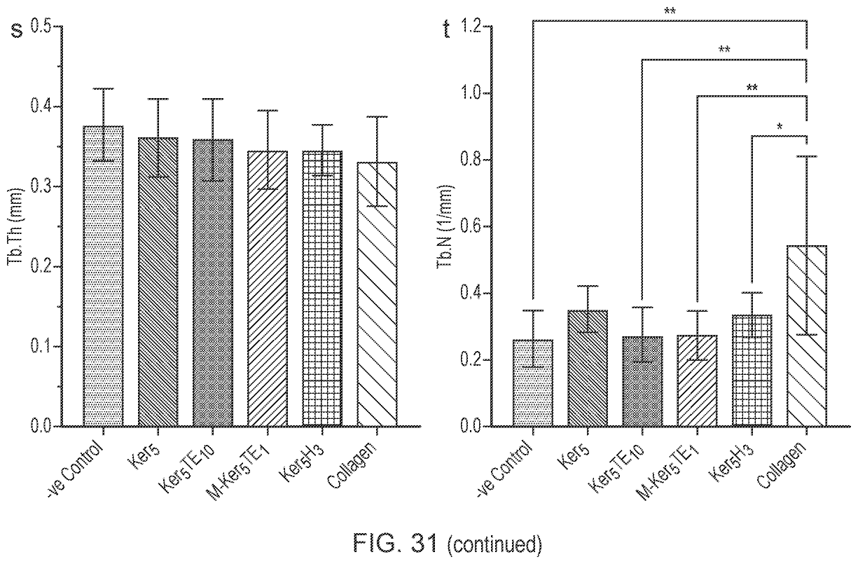

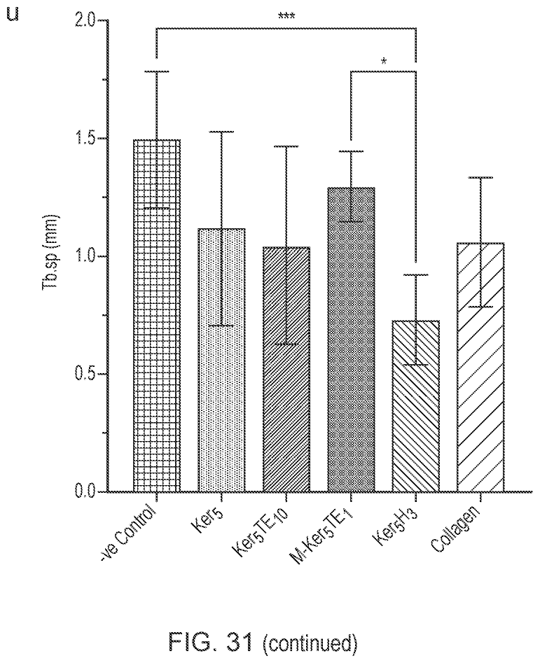

FIG. 31a-c show the round segmental calvarial defect in the rats.

FIG. 31d shows the hydrated Keratin membranes as implanted, covering the defect.

The rats were sacrificed after 8-weeks by carbon dioxide (CO₂) overdose. Decapitation was performed to all animals' postmortem, and skulls were submerged in 10% neutral buffered formaldehyde. Assessment of the biocompatibility and bone healing potential of these membranes was achieved by X-ray Radiography and micro-Computed Tomography.

To generate x-ray scans of the calvaria, the sample were exposed at 60 kVp and 7 mA for 6 s using 2D intraoral X-ray unit.

FIG. 31e-j—show X-ray scans of the different experimental groups. All of the experimental groups show effective bone healing as compared with the negative control.

To perform micro-Computed Tomography (Micro-CT), a high-resolution Micro-CT system was used to generate a three-dimensional image for the scanned specimens. The X-ray generator was operated at an accelerated potential of 81 kV with a current of 123 μA. The image resolution was 9 μm. The following parameters were assessed: bone volume [BV (mm2)], ratio of bone volume to trabecular volume [BV/TV (%)], trabecular thickness Tb. Th (mm), trabecular number T.N (mm−1), and trabecular separation T.Sp (mm).

FIG. 31j-u—show micro-Computed Tomography (Micro-CT) scans and analysis. All of the experimental groups show effective bone healing as compared with the negative control.

### REFERENCES

1. Palmer, L. C., Newcomb, C. J., Kaltz, S. R., Spoerke, E. D., & Stupp, S. I. Biomimetic systems for hydroxyapatite mineralization inspired by bone and enamel. Chem. Rev., 108, 4754-4783.
2. B. Marie, C. Joubert, A. Tayale, I. Zanella-Cleon, C. Belliard, D. Piquemal, N. Cochennec-Laureau, F. Marin, Y. Gueguen, C. Montagnani, Different secretory repertoires control the biomineralization processes of prism and nacre deposition of the pearl oyster shell. Proc. Natl. Acad. Sci. U.S.A. 109, 20986-20991 (2012).
3. Addadi, L. & Weiner, S. Control and design principles in biological mineralization. Angew. Chem. Int. Ed. Engl. 31, 153-169 (1992).
4. Kirkham J, Firth A, Vernals D, Boden N, Robinson C, Shore R C, et al. Self-assembling peptide scaffolds promote enamel remineralization. J Dent Res. 2007; 86(5):426-30.
5. Chen H, Tang Z, Liu J, Sun K, Chang SR, Peters M C, et al. Acellular synthesis of a human enamel-like microstructure. Advanced Materials. 2006; 18(14):1846-51.
6. Yamagishi, K. et al. A synthetic enamel for rapid tooth repair. Nature 433, 819 (2005).
7. Yin, Y., Yun, S., Fang, J. & Chen, H. Chemical regeneration of human tooth enamel under near-physiological conditions. Chem. Commun. 5892-5894 (2009).
8. Shao, C., Jin, B., Mu, Z., Lu, H., Zhao, Y., Wu, Z., Yan, L., Zhang, Z., Zhou, Y., Pan, H., Liu, Z., and Tang, R. Repair of tooth enamel by a biomimetic mineralization frontier ensuring epitaxial growth. Science Advances, 5, Eaaw9569 (2019).
9. Ruan, Q., Zhang, Y., Yang, X., Nutt, S. & Moradian-Oldak, J. An amelogeninchitosan matrix promotes assembly of an enamel-like layer with a dense interface. Acta Biomater. 9, 7289-7297 (2013).
10. Elsharkawy, S., Al-Jawad, M., Pantano M. F., Tejeda-Montes, E., Mehta, K., Jamal, H., Agarwal, S., Shuturminska, K., Rice, A., Tarakina, N. V., Wilson, R. M., Bushby, A.J., Alonso, M., Rodriguez-Cabello, J. C., Barbieri E., Hernández A. d. R., Stevens M. M., Pugno N. M., Anderson, P. & Alvaro Mata. Protein disorder-order interplay to guide the growth of hierarchical mineralized structures. Nature Communications, 9, 2145 (2018).
11. Boskey, A. L., & Villarreal-Ramirez, E. Intrinsically disordered proteins and biomineralization. Matrix biology: j.matbio., 52-54, 43-59 (2016).
12. Beniash, E., Simmer, J. P. & Margolis, H. C. Structural changes in amelogenin upon self-assembly and mineral interactions. J. Dent. Res. 91, 967-972 (2012).
13. Fincham, A. G. et al. Evidence for amelogenin ‘nanospheres’ as functional components of secretory-stage enamel matrix. J. Struct. Biol. 115, 50-59 (1995).
14. Carneiro, K. M. M. et al. Amyloid-like ribbons of amelogenins in enamel mineralization. Sci. Rep. 6, 23105 (2016).
15. Busch, S., Schwarz, U. & Kniep, R. Morphogenesis and structure of human teeth in relation to biomimetically grown fluorapatite-gelatine composites. Chem. Mater. 13, 3260-3271 (2001).
16. Delak, K. et al. The tooth enamel protein, porcine amelogenin, is an intrinsically disordered protein with an extended molecular configuration in the monomeric form. Biochemistry 48, 2272-2281 (2009).
17. Coulombe P. A., Omary M. B. ‘Hard’ and ‘soft’ principles defining the structure, function and regulation of keratin intermediate filaments. Curr. Opin. Cell. Biol. 14:110-22 (2002).

1. A method of producing a keratin composition, particularly a keratin membrane, comprising the steps of:
(a) Providing a keratin solution comprising keratin in a solvent;
(b) Casting the solution on a substrate; and
(c) Allowing the solution to dry.

2. A keratin solution comprising at least one keratin in a solvent.

3. A keratin composition, such as a membrane, comprising a keratin.

4. A keratin composition according to claim 3, wherein the composition comprises keratin spherulites.

5. A mineralised keratin composition comprising the composition of claim 3 or claim 4.

6. A method or solution according to claim 1 or 2 wherein the solution comprises at least two keratins.

7. A method, solution or composition according to any preceding claim wherein keratin or keratins are wool keratins.

8. A method, solution or composition according to any preceding claim, wherein when the composition comprises more than one keratin, the lightest keratin present in the solution or composition has a molecular weight that is less than 60% of the molecular weight of the heaviest keratin present in the solution or structure.

9. A method, solution or composition according to any preceding claim, wherein the keratins are highly negative.

10. A method, solution or composition according to any preceding claim, wherein at least one, or both, several or all of the keratins are alpha-keratins.

11. A method, solution or composition according to any preceding claim, wherein at least one, or both, several or all of the keratins are high sulphur keratins.

12. A method, solution or composition according to any of claims 1 to 10, wherein at least one, or both, several or all of the keratins are low sulphur keratins.

13. A method, solution or composition according to any of claims 1 to 10, wherein the solution or composition comprises both high and low sulphur keratins.

14. A method, solution or composition according to any of claims 1 to 5, wherein the solution or composition comprises one keratin, and wherein the keratin is not KRT75.

15. A method or solution according to any of claims 1, 2 or 6 to 14, wherein the solvent used is water, one or more polar aprotic solvent, or one or more alcohol, or a combination thereof.

16. A method or solution according to any of claims 1, 2 or 6 to 15, wherein, wherein the solvent comprises or is selected from water, ethanol, water and ethanol, water and DMF, DMSO, DMSO and DMF.

17. A method or solution according to any of claims 1, 2 or 6 to 16, wherein the solvent is deionised and/or comprise essentially no solutes other than keratin.

18. A method or solution according to any of claims 1, 2 or 6 to 17, wherein, wherein the solution comprises salts such as sodium, sulphate, chloride, zinc, carbonate, phosphate, calcium, fluoride, iron or potassium may be added to the solvent.

19. A method or solution according to any of claims 1, 2 or 6 to 18, wherein the pH of the solvent is between 3 and 12.

20. A method or solution according to any of claims 1, 2 or 6 to 19, wherein the concentration of the keratin in the solution is between 1 and 25 wt %.

21. A method according to any of claims 1 and 6 to 20, wherein the substrate is at least slightly hydrophobic.

22. A method according to any of claims 1 and 6 to 21, wherein the substrate is a polymeric organosilicon compound, such as polymethylesiloxane or polydimethylsiloxane.

23. A method according to any of claims 1 and 6 to 21, wherein the substrate is a tooth or teeth, or a bone.

24. A method according to any of claims 1 and 6 to 23, wherein the keratin solution is dried for at least 30 seconds.

25. A method according to any of claims 1 and 6 to 24, wherein the keratin solution is dried at a temperature between 15° C. and 50° C.

26. A method or composition according to any preceding claim, wherein the solution comprises a cross-linker, such as triethylene glycol-dimethacrylate (TEGDMA), hexamethylene diisocyantate (HDI) and/or poly-ethylene glycol dimethacrylate (PEGDMA).

27. A method or composition according to claim 26, wherein the cross-linker comprises a photo-initiator.

28. A method according to claim 27, further comprising the step of directing light at the solution, after casting it, in order to photo-initiate membrane formation.

29. A keratin composition according to claim 4 to 20, 26 or 27, wherein the spherulites are generally between 3 and 50 μm in diameter.

30. A keratin composition according to claim 4 to 20, 26, 27 or 29, wherein the composition is in the form of a membrane

31. A keratin composition according to claim 30, wherein the membrane is at least 10, 15, 20, 25 or 30 μm thick.

32. A keratin composition according to claim 30 or 31, wherein the membrane is less than 230, 225, 220, 215, 210, 205 or 200 μm thick.

33. A method according to any of claims 1 or 6 to 28, wherein the step of providing the keratin solution comprises the step of dissolving the keratin in the solvent.

34. A method according to any of claim 1, 6 to 28 or 33, further comprising the step of washing the composition, especially in water, and/or storing the composition, especially in water.

35. A method according to any of claim 1, 6 to 28, 33 or 34 further comprising the step of incubating the keratin composition in a mineralisation solution.

36. A method according to claim 35, wherein the mineralisation solution comprises calcium or a sodium salt or both.

37. A method according to claim 35 or 36 further comprising the step of adding an acid, such as nitric acid, hydrochloric acid or phosphoric acid, to the mineralisation solution.

38. A method according to claim 35, 36 or 37, wherein the mineralisation solution is, or comprises a bodily fluid, such as saliva, blood, interstitial fluid, serum or plasma.

39. A method or composition according to any preceding claim, wherein the keratins are obtained or obtainable by extracting keratin(s) from animal or human hair, fur, wool, hooves, shells, horns, claws, nails, quills or feathers.

40. A method of extracting keratin from animal or human tissue, such as hair, fur, wool, hooves, shells, horns, claws, nails, quills or feathers, particularly wool comprising the steps of:
(a) Mixing the tissue with an extraction solution;
(b) Heating the mixture;
(c) Separating a keratin containing supernatant from the remainder of the mixture.

41. The method of claim 40, wherein the extraction solution comprises one or more of urea, sodium dodecyl sulfate, 2-mercaptoethanol, thiourea dithiothreitol (DTT), [3-Cholamidopropyl)dimethylammonio]-1-propanesulfonate (CHAPS), L-cysteine, ampholytes, glycerol, triton X-100, sodium phosphate, sodium metabisulfite, sodium hydroxide, peracetic acid, 1-butyl-3-methylimidazolium chloride (BMIM+Cl₂), iodoacetate, zinc acetate, ethylenediaminetetraacetic acid (EDTA), Tris-HCl buffer, HEPES buffer and other buffers.

42. The method of claim 40 or 41, wherein the mixture is heated at between 4° and 60° C., for around 8 hours.

43. The method of any of claims 40 to 42, further comprising the step of drying the keratin solution.

44. A keratin solution, composition or mineralised composition obtainable or obtained by the method of any of claims 1, 6 to 28 or 33 to 39.

45. A pharmaceutical composition comprising a keratin solution, keratin composition or a mineralised keratin composition according to any of claim 2 to 20, 26, 27, 29 to 32, 39 or 44.

46. The pharmaceutical composition of claim 45, wherein the composition is in the form of or added to a hydrogel, a solution, a paint, a varnish, a coating, a scaffold, such as a 3D printed scaffold, a membrane, a toothpaste, a strip, particularly a dental strip, a dental restorative material, a composite, a gum, a metallic implant, a cement, a ceramic, a paste, a malleable putty, a film or a dentine desensitizing agent.

47. A medical structure or device such as a synthetic graft, a prosthesis or orthosis, an implant or a tissue graft, particularly a dental or bone graft, comprising a keratin solution, a keratin composition, or a mineralised keratin composition or a pharmaceutical composition according to any of claims 2 to 20, 26, 27, 29 to 32, 39, or 44 to 46.

48. A keratin solution, a keratin composition, a mineralised keratin composition, a pharmaceutical composition, a structure or device according to any of claims 2 to 20, 26, 27, 29 to 32, 39, or 44 to 47, for use in therapy.

49. The solution, composition, structure or device according to any of claims 2 to 20, 26, 27, 29 to 32, 39, or 44 to 47 for use in dental treatments, particularly for use in the treatment of white spot lesions (WSLs) or dental caries.

50. The solution, composition, structure or device according to any of claims 2 to 20, 26, 27, 29 to 32, 39, or 44 to 47 for use in the treatment or prevention of teeth demineralization, dental erosion, dental abrasion, alveolar bone erosion, periodontitis, peri-implantitis or dental pulp disease, dental hypersensitivity and soft tissue repair e.g., grafting and augmentation.

51. The solution, composition, structure or device according to any of claims 2 to 20, 26, 27, 29 to 32, 39, or 44 to 47 for use in bone repair, healing or regeneration.

52. A method of treating WSLs or dental caries, or bone damage or disorders, comprising administering a keratin composition, or a mineralised keratin composition or a pharmaceutical composition according to any of claims 2 to 20, 26, 27, 29 to 32, 39, or 44 to 46, or utilising a device according to claim 47.

53. A product or kit comprising (a) a keratin solution or composition and (b) a further mineralisation solution and/or crosslinker.

54. A kit comprising a keratin solution, keratin composition or pharmaceutical composition according to any of claims 2 to 20, 26, 27, 29 to 32, 39, or 44 to 46 and an applicator for applying the solution or composition to the site of interest.
